(19) United States
(12) Patent Application Publication
(10) Pub. No.: US 2026/0256768 A1
(43) Pub. Date: Sep. 3, 2026

(54) DRY POWDER COMPOSITIONS OF BEDAQUILINE AND SALTS AND METHODS OF USE THEREOF

(71) Applicant: Insmed Incorporated, Bridgewater, NJ (US)

(72) Inventors: Sachin GHARSE, Bridgewater, NJ (US); Amruta SABNIS, Bridgewater, NJ (US); Donna KONICEK, Bridgewater, NJ (US); Adam PLAUNT, Bridgewater, NJ (US); Sasha ROSE, Bridgewater, NJ (US); Vladimir MALININ, Bridgewater, NJ (US); Walter PERKINS, Bridgewater, NJ (US)

(21) Appl. No.: 18/836,285

(22) PCT Filed: Feb. 6, 2023
(86) PCT No.: PCT/US2023/062038

§ 371 Date: Aug. 6, 2024

Related U.S. Application Data
(60) Provisional application No. 63/307,373, filed on Feb. 7, 2022.

(51) Int. Cl. A61K 31/47; A61K 9/00; A61K 9/16

(52) U.S. Cl. CPC A61K 31/47; A61K 9/0075; A61K 9/1617

(57) ABSTRACT

The present disclosure provides dry powder compositions comprising bedaquiline, and/or a pharmaceutically acceptable salt thereof, and methods of administering the same to a patient to treat mycobacterial lung diseases, e.g., tuberculosis and nontuberculous mycobacterial (NTM) lung diseases, as well as lung cancer or a metastatic cancer to the lung by inhalation via a dry powder inhaler.

### CROSS REFERENCE TO RELATED APPLICATION

This application claims priority from U.S. Provisional Application Ser. No. 63/307,373, filed Feb. 7, 2022, the disclosure of which is incorporated by reference herein in its entirety.

### BACKGROUND OF THE INVENTION

Bedaquiline (IUPAC name: [(1R,2S)-1-(6-bromo-2-methoxyquinolin-3-yl)-4-(dimethylamino)-2-naphthalen-1-yl-1-phenylbutan-2-ol]) and its pharmaceutically acceptable salts, e.g., bedaquiline fumarate, are mycobacterial inhibitors that have been used historically for treating mycobacterial diseases, e.g., diseases caused by pathogenic mycobacteria such as Mycobacterium tuberculosis, Mycobacterium bovis, Mycobacterium avium, and Mycobacterium marinum. See U.S. Pat. Nos. 7,498,343 and 8,546,428, the disclosure of each of which is incorporated herein by reference in their entireties for all purposes. Bedaquiline fumarate, marketed under the brand name SIRTURO®, has been approved by the U.S. Food and Drug Administration as part of combination therapy in adult and pediatric patients with pulmonary multi-drug resistant tuberculosis (MDR-TB).

More recently, bedaquiline has been shown to inhibit lung tumor growth and angiogenesis in an animal model. Wu et al., Biochemical and Biophysical Research Communications, 495:267-272 (2018); incorporated by reference in its entirety for all purposes. Similarly, bedaquiline was also shown to have cytotoxic properties against two non-small cell lung cancer cell lines. Parvathaneni et al., Int J Mol Sci. 22(9): 4783 (2021); incorporated by reference in its entirety for all purposes. Additionally, bedaquiline was shown to block the propagation and expansion of MCF7 breast cancer cells-derived cancer stem-like cells, as well as inhibit cancer metastasis in vivo. Fiorillo M et al., Aging (Albany NY). 8(8):1593-607 (2016); Fiorillo M et al., Cell Death Differ 28:2797-2817 (2021); each of which is incorporated by reference in its entirety for all purposes.

The present disclosure provides dry powder compositions comprising bedaquiline and/or one or more of pharmaceutically acceptable salts of bedaquiline useful for pulmonary administration, and methods for administering the same to patients in need of treatment for mycobacterial lung diseases, e.g., tuberculosis and nontuberculous mycobacterial (NTM) lung diseases, as well as patients in need of treatment for lung cancer or a metastatic cancer to the lung.

### SUMMARY OF THE INVENTION

In a first aspect, the present disclosure provides a dry powder composition comprising:
(a) from about 65 wt % to about 95 wt % of bedaquiline, and/or a pharmaceutically acceptable salt thereof; and
(b) the balance being trileucine;
wherein the entirety of (a) and (b) is 100 wt %.

In one embodiment of the dry powder composition, (a) is bedaquiline. In another embodiment, (a) is a pharmaceutically acceptable salt of bedaquiline. In another embodiment, (a) is a combination of bedaquiline and a pharmaceutically acceptable salt thereof.

In one embodiment of the dry powder composition, the pharmaceutically acceptable salt of bedaquiline is a pharmaceutically acceptable acid addition salt of bedaquiline, e.g., a fumarate salt. In one embodiment, the pharmaceutically acceptable salt of bedaquiline comprises a fumarate salt of bedaquiline. In another embodiment, the pharmaceutically acceptable salt of bedaquiline is a fumarate salt of bedaquiline. The fumarate salt of bedaquiline, in one embodiment, is (αS, βR)-6-bromo-α-[2-(dimethylamino)ethyl]-2-methoxy-α-1-naphthalenyl-β-phenyl-3-quinolineethanol (2E)-2-butenedioate (1:1) represented by the following formula:

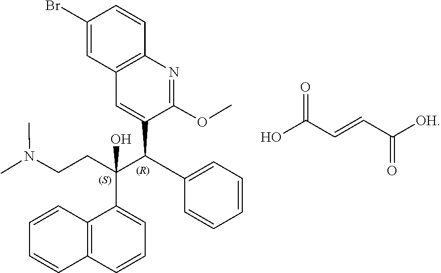

In one embodiment of the dry powder composition, bedaquiline, and/or a pharmaceutically acceptable salt thereof is present at from about 77 wt % to about 93 wt %, from about 80 wt % to about 90 wt %, from about 79 wt % to about 81 wt %, from about 89 wt % to about 91 wt %, about 80 wt %, or about 90 wt %, of the total weight of the dry powder composition.

In another aspect of the present disclosure, a method for treating a mycobacterial lung disease in a patient in need of treatment is provided. The method includes administering an effective amount of a dry powder composition disclosed herein to the lungs of the patient by inhalation via a dry powder inhaler (DPI).

In one embodiment, the mycobacterial lung disease is caused by Mycobacterium tuberculosis, Mycobacterium bovis, or a combination thereof. In a further embodiment, the mycobacterial lung disease is caused by Mycobacterium tuberculosis. In a further embodiment, the dry powder composition administered comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

In another embodiment, the mycobacterial lung disease is a nontuberculous mycobacterial (NTM) lung disease caused by, e.g., Mycobacterium abscessus, Mycobacterium asiaticum, Mycobacterium avium, Mycobacterium avium complex (MAC) (Mycobacterium avium and Mycobacterium intracellulare), Mycobacterium avium subsp. hominissuis (MAH), Mycobacterium bolletii, Mycobacterium celatum, Mycobacterium chelonae, Mycobacterium conspicuum, Mycobacterium fortuitum, Mycobacterium fortuitum complex (Mycobacterium fortuitum and Mycobacterium chelonae), Mycobacterium genavense, Mycobacterium gordonae, Mycobacterium haemophilum, Mycobacterium immunogenum, Mycobacterium kansasii, Mycobacterium lentiflavum, Mycobacterium malmoense, Mycobacterium marinum, Mycobacterium mucogenicum, Mycobacterium nonchromogenicum, Mycobacterium peregrinum, Mycobacterium scrofulaceum, Mycobacterium shimoidei, Mycobacterium simiae, Mycobacterium smegmatis, Mycobacterium szulgai, Mycobacterium terrae, Mycobacterium terrae complex, Mycobacterium triplex, Mycobacterium ulcerans, Mycobacterium xenopi, or a combination thereof. In a further embodiment, the dry powder composition administered comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

In another aspect of the present disclosure, a method for treating lung cancer in a patient in need of treatment is provided. The method includes administering an effective amount of a dry powder composition disclosed herein to the lungs of the patient by inhalation via a DPI. In one embodiment, the lung cancer is non-small cell lung cancer. In another embodiment, the lung cancer is small cell lung cancer. In another embodiment, the lung cancer comprises a lung carcinoid tumor, an adenoid cystic carcinoma, a lymphoma, a sarcoma, a hamartoma, or a combination of the foregoing. In one embodiment, the dry powder composition administered comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

In another aspect of the present disclosure, a method for treating a metastatic cancer to the lung, i.e., a cancer that spreads to the lung(s), in a patient in need of treatment is provided. The method includes administering an effective amount of a dry powder composition disclosed herein to the lungs of the patient by inhalation via a DPI. In some embodiments, the metastatic cancer to the lung comprises metastasis of bladder cancer to the lung, metastasis of bone cancer to the lung, metastasis of breast cancer to the lung, metastasis of colorectal cancer to the lung, metastasis of hematopoietic cancer to the lung, metastasis of kidney cancer to the lung, metastasis of leukemia to the lung, metastasis of liver cancer to the lung, metastasis of ovarian cancer to the lung, metastasis of pancreatic cancer to the lung, metastasis of prostate cancer to the lung, metastasis of skin cancer, e.g., melanoma, to the lung, metastasis of stomach cancer to the lung, or metastasis of thyroid cancer to the lung, In one embodiment, the dry powder composition administered comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

### BRIEF DESCRIPTION OF THE FIGURES

FIG. 1A is a graph showing distribution of the bedaquiline fumarate and trileucine components of dry powder composition BDFDP1 in the impactor stages.

FIG. 1B is a graph showing distribution of the bedaquiline fumarate and trileucine components of dry powder composition BDFDP2 in the impactor stages.

FIG. 2A is a graph showing distribution of the bedaquiline fumarate and trileucine components of dry powder composition BDFDP4 in the impactor stages.

FIG. 2B is a graph showing distribution of the bedaquiline fumarate and trileucine components of dry powder composition BDFDP5 in the impactor stages.

FIG. 3A is a graph showing deposition of the bedaquiline fumarate component of dry powder composition BDFDP6 containing 10% trileucine and dry powder composition BDFDP8 containing 20% trileucine in the NGI at t=0.

FIG. 3B is a graph showing deposition of the trileucine component of dry powder composition BDFDP6 containing 10% trileucine and dry powder composition BDFDP8 containing 20% trileucine in the NGI at t=0.

FIG. 4 is a graph showing deposition of the bedaquiline fumarate component of dry powder composition BDFDP8 containing 20% trileucine and dry powder composition BDFDP17 containing 20% leucine in the NGI at t=0.

FIG. 5A is a graph showing deposition of the bedaquiline fumarate component of dry powder composition BDFDP8 containing 20% trileucine in the NGI at t=0 and t=2 months.

FIG. 5B is a graph showing deposition of the trileucine component of dry powder composition BDFDP8 containing 20% trileucine in the NGI at t=0 and t=2 months.

FIG. 6A is a graph showing deposition of the bedaquiline fumarate component of dry powder composition BDFDP8 containing 20% trileucine in the NGI at t=0 (left bar in each data set for each NGI component), t=6 months (middle bar in each data set for each NGI component), and t=12 months (right bar in each data set for each NGI component).

FIG. 6B is a graph showing deposition of the bedaquiline fumarate component of dry powder composition BDFDP17 containing 20% leucine in the NGI at t=0 (left bar in each data set for each NGI component), t=6 months (middle bar in each data set for each NGI component), and t=12 months (right bar in each data set for each NGI component).

FIG. 7A is a graph showing lung PK data following administration to healthy rats of a single dose of bedaquiline fumarate dry powder composition BDFDP8 containing 20% trileucine, or dry powder composition BDFDP17 containing 20% leucine by nose-only inhalation, or a single dose of a bedaquiline fumarate solution by oral gavage.

FIG. 7B is a graph showing plasma PK data following administration to healthy rats of a single dose of bedaquiline fumarate dry powder composition BDFDP8 containing 20% trileucine, or dry powder composition BDFDP17 containing 20% leucine by nose-only inhalation, or a single dose of a bedaquiline fumarate solution by oral gavage.

### DETAILED DESCRIPTION OF THE INVENTION

Throughout the present disclosure, the term “about” may be used in conjunction with numerical values and/or ranges. The term “about” is understood to mean those values near to a recited value. For example, “about 40 [units]” may mean within ±25% of 40 (e.g., from 30 to 50), within ±20%, ±15%, ±10%, ±9%, ±8%, ±7%, ±6%, ±5%, ±4%, ±3%, ±2%, ±1%, less than ±1%, or any other value or range of values therein or there below.

A “pharmaceutically acceptable salt” as used herein can refer to a pharmaceutically acceptable acid addition or base addition salt. In one embodiment, a “pharmaceutically acceptable salt” refers to a pharmaceutically acceptable acid addition salt, i.e., a salt which retains the biological effectiveness and properties of the free base, which is not biologically or otherwise undesirable, and which is formed with an inorganic acid such as, but not limited to, hydrochloric acid (HCl), hydrobromic acid, sulfuric acid, nitric acid, phosphoric acid and the like, or an organic acid such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, hydroxyacetic acid, isobutyric acid, lactic acid (e.g., as lactate), lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propanoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid (TFA), undecylenic acid, and the like. In one embodiment, the pharmaceutically acceptable salt comprises one pharmaceutically acceptable acid addition salt, e.g., a fumarate salt. In another embodiment, the pharmaceutically acceptable salt comprises two or more pharmaceutically acceptable acid addition salts. In a further embodiment, the two or more pharmaceutically acceptable acid addition salts include a fumarate salt.

In another embodiment, a “pharmaceutically acceptable salt” refers to a pharmaceutically acceptable base addition salt, which retains the biological effectiveness and properties of the free acid, and which is not biologically or otherwise undesirable. A pharmaceutically acceptable base addition salt is prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. In one embodiment, inorganic salts include the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, hybramine, theobromine, triethanolamine, tromethamine, purines, piperazine, piperidine, N-ethylpiperidine, polyamine resins and the like. Organic bases that can be used to form a pharmaceutically acceptable salt include isopropylamine, diethylamine, ethanolamine, trimethylamine, dicyclohexylamine, choline and caffeine. In one embodiment, the pharmaceutically acceptable salt comprises one pharmaceutically acceptable base addition salt. In another embodiment, the pharmaceutically acceptable salt comprises two or more pharmaceutically acceptable base addition salts.

Throughout the present specification, numerical ranges are provided for certain quantities. It is to be understood that these ranges comprise all subranges therein. Thus, the range “50-80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “treating” in one embodiment, includes: (1) preventing or delaying the appearance of clinical symptoms of the state, disorder or condition developing in the subject that may be afflicted with or predisposed to the state, disorder or condition but does not yet experience or display clinical or subclinical symptoms of the state, disorder or condition; (2) inhibiting the state, disorder or condition (e.g., arresting, reducing or delaying the development of the disease, or a relapse thereof in case of maintenance treatment, of at least one clinical or subclinical symptom thereof); and/or (3) relieving the condition (e.g., causing regression of the state, disorder or condition or at least one of its clinical or subclinical symptoms). In one embodiment, “treating” refers to inhibiting the state, disorder or condition (e.g., arresting, reducing or delaying the development of the disease, or a relapse thereof in case of maintenance treatment, of at least one clinical or subclinical symptom thereof). In another embodiment, “treating” refers to relieving the condition (for example, by causing regression of the state, disorder or condition or at least one of its clinical or subclinical symptoms). The benefit to a subject to be treated is either statistically significant as compared to the state or condition of the same subject before the treatment, or as compared to the state or condition of an untreated control subject, or the benefit is at least perceptible to the subject or to the physician.

“Effective amount” means an amount of a dry powder composition or the active pharmaceutical ingredient (API) in the dry powder composition, e.g., bedaquiline, a pharmaceutically acceptable salt of bedaquiline, or a combination of bedaquiline and its pharmaceutically acceptable salt(s), of the present disclosure that is sufficient to result in the desired therapeutic response.

The present disclosure relates to dry powder compositions comprising bedaquiline, and/or a pharmaceutically acceptable salt thereof. The dry powder compositions are used in certain aspects, in methods for treating a mycobacterial lung disease in a patient in need of treatment by administering an effective amount of a dry powder composition disclosed herein to the lungs of the patient by inhalation via a dry powder inhaler (DPI). In another aspect, a method is disclosed for treating lung cancer in a patient in need of treatment. The method, as described herein, comprises administering to a patient in need of treatment, an effective amount of a dry powder composition disclosed herein to the lungs of the patient by inhalation via a DPI. In still another aspect, a method is disclosed for treating a metastatic cancer to the lung in a patient in need of treatment. The method, as described herein, comprises administering to a patient in need of treatment, an effective amount of a dry powder composition disclosed herein to the lungs of the patient by inhalation via a DPI.

In a first aspect, the present disclosure provides a dry powder composition comprising:
(a) from about 65 wt % to about 95 wt % of bedaquiline, and/or a pharmaceutically acceptable salt thereof; and
(b) the balance being trileucine;
wherein the entirety of (a) and (b) is 100 wt %.

In one embodiment of the dry powder composition, (a) is bedaquiline. In another embodiment, (a) is a pharmaceutically acceptable salt of bedaquiline. In another embodiment, (a) is a combination of bedaquiline and a pharmaceutically acceptable salt thereof.

In one embodiment of the dry powder composition, the pharmaceutically acceptable salt of bedaquiline is a pharmaceutically acceptable acid addition salt of bedaquiline, e.g., a fumaric acid addition salt, i.e., a fumarate salt. In one embodiment, the pharmaceutically acceptable salt of bedaquiline comprises a fumarate salt of bedaquiline. In another embodiment, the pharmaceutically acceptable salt of bedaquiline is a fumarate salt of bedaquiline. The fumarate salt of bedaquiline, in one embodiment, is (αS, βR)-6-bromo-α-[2-(dimethylamino)ethyl]-2-methoxy-α-1-naphthalenyl-β-phenyl-3-quinolineethanol (2E)-2-butenedioate (1:1) represented by the following formula:

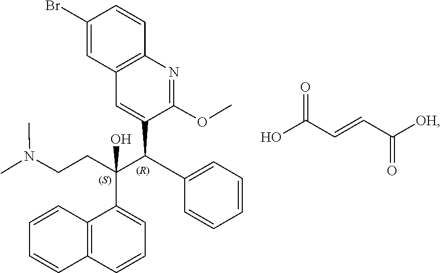

which is disclosed in U.S. Pat. No. 8,546,428, incorporated herein by reference in its entirety for all purposes.

Exemplary embodiments of the dry powder composition comprising various amounts of (a) bedaquiline, and/or a pharmaceutically acceptable salt thereof, and (b) trileucine, expressed as wt % of the total weight of the dry powder composition, are provided in Table 1 below.

TABLE 1
Exemplary embodiments of the dry powder composition
comprising (a) bedaquiline, and/or a pharmaceutically
acceptable salt thereof, and (b) trileucine

| (a) Wt % of bedaquiline, and/or a | (b) Wt % of |
|---|---|
| pharmaceutically acceptable salt thereof | trileucine |
| a = From about 68 to about 95 | b = 100-a |
| a = From about 70 to about 95 | b = 100-a |
| a = From about 73 to about 95 | b = 100-a |
| a = From about 77 to about 93 | b = 100-a |
| a = From about 80 to about 90 | b = 100-a |
| a = From about 85 to about 89 | b = 100-a |
| a = From about 75 to about 85 | b = 100-a |
| a = From about 78 to about 82 | b = 100-a |
| a = From about 79 to about 81 | b = 100-a |
| a = About 80 | b = 100-a |
| | (i.e., about 20) |
| a = From about 85 to about 95 | b = 100-a |
| a = From about 88 to about 92 | b = 100-a |
| a = From about 89 to about 91 | b = 100-a |
| a = About 90 | b = 100-a |
| | (i.e., about 10) |

Mass median aerodynamic diameter (MMAD) is the value of aerodynamic diameter for which 50% of the mass in a given aerosol is associated with particles smaller than the median aerodynamic diameter (MAD), and 50% of the mass is associated with particles larger than the MAD. MMAD can be determined by impactor measurements, e.g., the Andersen Cascade Impactor (ACI) or the Next Generation Impactor (NGI). In one embodiment, the dry powder composition is in the form of an aerosol comprising particles with an MMAD of from about 1 μm to about 3 μm, as measured by NGI. In another embodiment, the dry powder composition is in the form of an aerosol comprising particles with an MMAD of from about 1 μm to about 2 μm, as measured by NGI. In still another embodiment, the dry powder composition is in the form of an aerosol comprising particles with an MMAD of from about 1.5 μm to about 2 μm, as measured by NGI. In still another embodiment, the dry powder composition is in the form of an aerosol comprising particles with an MMAD of from about 1.5 μm to about 1.8 μm, as measured by NGI. In still another embodiment, the dry powder composition is in the form of an aerosol comprising particles with an MMAD of about 1.6 μm, as measured by NGI.

“Fine particle fraction” or “FPF” refers to the fraction of an aerosol having a particle size less than 5 μm in diameter, as measured by cascade impaction. FPF is usually expressed as a percentage. FPF has been demonstrated to correlate to the fraction of the powder that is deposited in the lungs of the patient. In one embodiment, the dry powder composition is in the form of an aerosol comprising particles with an FPF of from about 50% to about 75%, as measured by NGI. In another embodiment, the dry powder composition is in the form of an aerosol comprising particles with an FPF of from about 60% to about 75%, as measured by NGI. In another embodiment, the dry powder composition is in the form of an aerosol comprising particles with an FPF of from about 65% to about 75%, as measured by NGI. In another embodiment, the dry powder composition is in the form of an aerosol comprising particles with an FPF of from about 68% to about 72%, as measured by NGI. In another embodiment, the dry powder composition is in the form of an aerosol comprising particles with an FPF of about 71%, as measured by NGI.

Tap density of a powder is the ratio of the mass of the powder to the volume occupied by the powder after it has been tapped for a defined period of time. The tap density of a powder represents its random dense packing. Tap density can be determined using the method of USP Bulk Density and Tapped Density, United States Pharmacopeia convention, Rockville, Md., 10th Supplement, 4950-4951, 1999. In some embodiments, the dry powder composition comprises particles having a tap density of from about 0.3 g/cm³ to about 0.6 g/cm³, or from about 0.4 g/cm³ to about 0.5 g/cm³.

The dry powder compositions of the present disclosure may be produced, in one embodiment, from liquid compositions using lyophilization or spray-drying techniques. When lyophilization is used, the lyophilized composition may be milled to obtain the finely divided dry powder containing particles within the desired size range described herein. When spray-drying is used, the process is carried out under conditions that result in a finely divided dry powder containing particles within the desired size range described herein. Exemplary methods of preparing dry powder forms of pharmaceutical compositions are disclosed in WO 1996/032149, WO 1997/041833, WO 1998/029096, and U.S. Pat. Nos. 5,976,574, 5,985,248, and 6,001,336; the disclosure of each of which is incorporated herein by reference in their entireties for all purposes. Exemplary spray drying methods are described in WO 2020/223237, and U.S. Pat. Nos. 6,848,197 and 8,197,845, the disclosure of each of which is incorporated herein by reference in their entireties for all purposes.

In some embodiments, the dry powder compositions of the present disclosure are prepared by the following process. A spray drying feed solution is prepared by dissolving the active pharmaceutical ingredient (API, i.e., bedaquiline, and/or a pharmaceutically acceptable salt thereof), and excipients, e.g., trileucine, in a binary solvent system containing 1-propanol and water, or containing ethanol and water. In the spray drying feed solution, the volume ratio of 1-propanol or ethanol to water may be from about 60 (1-propanol or ethanol): 40 (water) to about 90 (1-propanol or ethanol): 10 (water), or from about 70 (1-propanol or ethanol): 30 (water) to about 80 (1-propanol or ethanol): 20 (water). In one embodiment, the binary solvent system contains ethanol and water at the volume ratio of about 70 (ethanol):30 (water).

Spray drying is initiated by starting the drying gas flow and heating up the drying gas by setting the desired inlet temperature at, for example, from about 100° C. to about 140° C., or from about 105° C. to about 125° C. After the spray dryer outlet temperature reaches a suitable temperature, for example, at from about 60° C. to about 85° C., or from about 60° C. to about 70° C., the liquid skid inlet is set to allow blank solvents to be atomized with the aid of nitrogen into the spray dryer, and the system is allowed to stabilize. After the system stabilizes, the liquid skid inlet is switched to the feed solution prepared above and the process is continued till the feed solution runs out. Powder is collected over the entire duration of the feed solution spray drying. At the point when the feed solution runs out, the liquid skid inlet is switched back to blank solvents, which are allowed to spray for from about 10 to about 15 minutes. After spraying the blank solvents, the system is shut down by, for example, in the case of a Buchi B-290 spray dryer, shutting down the feed pump and heater, the drying gas and finally the aspirator.

In another aspect of the present disclosure, a method for treating a mycobacterial lung disease in a patient in need of treatment is provided. The method includes administering an effective amount of a dry powder composition disclosed herein to the lungs of the patient by inhalation via a dry powder inhaler (DPI).

In one embodiment of the method, the mycobacterial lung disease is caused by Mycobacterium tuberculosis, Mycobacterium bovis, or a combination thereof. In a further embodiment, the mycobacterial lung disease is caused by Mycobacterium tuberculosis. In a further embodiment, the dry powder composition administered according to the method comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

In another embodiment of the method, the mycobacterial lung disease is a nontuberculous mycobacterial (NTM) lung disease. The NTM lung disease may be caused by, for example, Mycobacterium abscessus, Mycobacterium asiaticum, Mycobacterium avium, Mycobacterium avium complex (MAC) (Mycobacterium avium and Mycobacterium intracellulare), Mycobacterium avium subsp. hominissuis (MAH), Mycobacterium bolletii, Mycobacterium celatum, Mycobacterium chelonae, Mycobacterium conspicuum, Mycobacterium fortuitum, Mycobacterium fortuitum complex (Mycobacterium fortuitum and Mycobacterium chelonae), Mycobacterium genavense, Mycobacterium gordonae, Mycobacterium haemophilu Mycobacterium immunogenum, Mycobacterium kansasii, Mycobacterium lentiflavum, Mycobacterium malmoense, Mycobacterium marinum, Mycobacterium mucogenicum, Mycobacterium nonchromogenicum, Mycobacterium peregrinum, Mycobacterium scrofulaceum, Mycobacterium shimoidei, Mycobacterium simiae, Mycobacterium smegmatis, Mycobacterium szulgai, Mycobacterium terrae, Mycobacterium terrae complex, Mycobacterium triplex, Mycobacterium ulcerans, Mycobacterium xenopi, or a combination thereof.

In one embodiment, the NTM lung disease is caused by Mycobacterium abscessus. In a further embodiment, the dry powder composition administered according to the method comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

In another embodiment, the NTM lung disease is caused by Mycobacterium avium. In a further embodiment, the dry powder composition administered according to the method comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

In another embodiment, the NTM lung disease is caused by Mycobacterium avium subsp. Hominissuis. In a further embodiment, the dry powder composition administered according to the method comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

In another embodiment, the NTM lung disease is caused by Mycobacterium avium complex (Mycobacterium avium and Mycobacterium intracellulare). In a further embodiment, the dry powder composition administered according to the method comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

In another aspect of the present disclosure, a method for treating lung cancer in a patient in need of treatment is provided. The method includes administering an effective amount of a dry powder composition disclosed herein to the lungs of the patient by inhalation via a DPI. In one embodiment of the method, the dry powder composition administered according to the method comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

According to the American Cancer Society, there are two main types of lung cancer: non-small cell lung cancer (NSCLC) and small cell lung cancer (SCLC). NSCLC accounts for about 80% to 85% of lung cancers and includes adenocarcinoma, squamous cell carcinoma, and large cell carcinoma as main subtypes, as well as other less common subtypes, such as adenosquamous carcinoma and sarcomatoid carcinoma. SCLC accounts for about 10% to 15% of all lung cancers. Both NSCLC and SCLC are treatable by the methods provided herein. Other types of lung cancer treatable by the methods provided herein include lung carcinoid tumors, adenoid cystic carcinomas, lymphomas, and sarcomas, as well as benign lung tumors such as hamartomas.

In one embodiment of the method, the lung cancer is non-small cell lung cancer. In a further embodiment, the dry powder composition administered according to the method comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

In one embodiment of the method, the lung cancer is small cell lung cancer. In a further embodiment, the dry powder composition administered according to the method comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

In one embodiment of the method, the lung cancer comprises a lung carcinoid tumor, an adenoid cystic carcinoma, a lymphoma, a sarcoma, a hamartoma, or a combination thereof. In a further embodiment, the dry powder composition administered according to the method comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

In another aspect of the present disclosure, a method for treating a metastatic cancer to the lung, i.e., a cancer that spreads to the lung(s), in a patient in need of treatment is provided. The method includes administering an effective amount of a dry powder composition disclosed herein to the lungs of the patient by inhalation via a DPI. In one embodiment of the method, the dry powder composition administered according to the method comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

In one embodiment of the method, the metastatic cancer to the lung comprises metastasis of bladder cancer to the lung. In a further embodiment, the dry powder composition administered according to the method comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

In one embodiment of the method, the metastatic cancer to the lung comprises metastasis of bone cancer to the lung. In a further embodiment, the dry powder composition administered according to the method comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

In one embodiment of the method, the metastatic cancer to the lung comprises metastasis of breast cancer to the lung. In a further embodiment, the dry powder composition administered according to the method comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

In one embodiment of the method, the metastatic cancer to the lung comprises metastasis of colorectal cancer to the lung. In a further embodiment, the dry powder composition administered according to the method comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

In one embodiment of the method, the metastatic cancer to the lung comprises metastasis of hematopoietic cancer to the lung. In a further embodiment, the dry powder composition administered according to the method comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

In one embodiment of the method, the metastatic cancer to the lung comprises metastasis of kidney cancer to the lung. In a further embodiment, the dry powder composition administered according to the method comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

In one embodiment of the method, the metastatic cancer to the lung comprises metastasis of leukemia to the lung. In a further embodiment, the dry powder composition administered according to the method comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

In one embodiment of the method, the metastatic cancer to the lung comprises metastasis of liver cancer to the lung. In a further embodiment, the dry powder composition administered according to the method comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

In one embodiment of the method, the metastatic cancer to the lung comprises metastasis of ovarian cancer to the lung. In a further embodiment, the dry powder composition administered according to the method comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

In one embodiment of the method, the metastatic cancer to the lung comprises metastasis of pancreatic cancer to the lung. In a further embodiment, the dry powder composition administered according to the method comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

In one embodiment of the method, the metastatic cancer to the lung comprises metastasis of prostate cancer to the lung. In a further embodiment, the dry powder composition administered according to the method comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

In one embodiment of the method, the metastatic cancer to the lung comprises metastasis of skin cancer, e.g., melanoma, to the lung. In a further embodiment, the dry powder composition administered according to the method comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

In one embodiment of the method, the metastatic cancer to the lung comprises metastasis of stomach cancer to the lung. In a further embodiment, the dry powder composition administered according to the method comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

In one embodiment of the method, the metastatic cancer to the lung comprises metastasis of thyroid cancer to the lung. In a further embodiment, the dry powder composition administered according to the method comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1).

In one embodiment of the treatment methods disclosed herein, the administering includes aerosolizing the dry powder composition via a DPI to provide an aerosolized dry powder composition, and administering the aerosolized dry powder composition to the lungs of the patient by inhalation via the DPI.

In one embodiment of the treatment methods disclosed herein, the DPI is a single dose dry powder inhaler. The unit dose of a dry powder composition used in a DPI device is often a dry powder blister disc of a hard capsule. Exemplary DPI devices suitable for delivering the dry powder compositions of the present disclosure include the devices described in the following paragraphs, as well as the DPIs described in U.S. Pat. Nos. 6,766,799, 7,278,425 and 8,496,002, the disclosure of each of which is herein incorporated by reference in their entireties for all purposes. Other exemplary DPIs for use with the methods provided herein are provided below.

The AIR® inhaler (Alkermes) includes a small, breath-activated system that delivers porous powder from a capsule. The porous particles have an aerodynamic diameter of 1-5 μm. See International Patent Application Publication Nos. WO 1999/066903 and WO 2000/010541, the disclosure of each of which is incorporated herein by reference in their entireties.

Aerolizer™ (Novartis) is a single dose dry powder inhaler. In this device, dry powder medicament is stored in a capsule and released by piercing the capsule wall with TEFLON-coated steel pins. See U.S. Pat. Nos. 6,488,027 and 3,991,761, the disclosure of each of which is incorporated herein by reference in their entireties.

BANG OLUFSEN provides a breath actuated inhaler using blister strips with up to sixty doses. The dose is made available only during the inhalation by a novel trigger mechanism. The device is equipped with a dose counter and can be disposed of after all doses have been used. See EP 1522325, the disclosure of which is incorporated herein by reference in its entirety.

Clickhaler® (Innovata PLC) is a large reservoir breath-activated multidose device. See U.S. Pat. No. 5,437,270, the disclosure of which is incorporated herein by reference in its entirety.

DirectHaler™ (Direct-Haler A/S) is a single dose, pre-metered, pre-filled, disposable DPI device made from polypropylene. See U.S. Pat. No. 5,797,392, the disclosure of which is incorporated herein by reference in its entirety.

Diskus™ (GlaxoSmithKline) is a disposable small DPI device that holds up to 60 doses contained in double foil blister strips to provide moisture protection. See GB2242134, the disclosure of which is incorporated herein by reference in its entirety.

Eclipse™ (Aventis) is a breath actuated re-usable capsule device capable of delivering up to 20 mg of a dry power composition. The powder is sucked from the capsule into a vortex chamber where a rotating ball assists in powder disaggregation as a subject inhales. See U.S. Pat. No. 6,230,707 and WO 1995/003846, the disclosure of each of which is incorporated herein by reference in their entireties.

Flexhaler® is a plastic breath-activated dry powder inhaler and is amenable for use with the dry powder compositions provided herein.

FlowCaps® (Hovione) is a capsule-based, re-fillable, re-usable passive dry-powder inhaler that holds up to 14 capsules. The inhaler itself is moisture-proof. See U.S. Pat. No. 5,673,686, the disclosure of which is incorporated herein by reference in its entirety.

Gyrohaler® (Vectura) is a passive disposable DPI containing a strip of blisters. See GB2407042, the disclosure of which is incorporated herein by reference in its entirety.

The HandiHaler® (Boehringer Ingelheim GmbH) is a single dose DPI device. It can deliver up to 30 mg of a dry powder composition in capsules. See International Patent Application Publication No. WO 2004/024156, the disclosure of which is incorporated herein by reference in its entirety.

MicroDose® DPI (Microdose Technologies) is a small electronic DPI device. It uses piezoelectric vibrator (ultrasonic frequencies) to disaggregate the drug powder in an aluminum blister (single or multiple dose). See U.S. Pat. No. 6,026,809, the disclosure of which is incorporated herein by reference in its entirety.

Nektar Dry Powder Inhaler® (Nektar) is a palm-sized and easy-to-use device. It provides convenient dosing from standard capsules and flow-rate-independent lung deposition.

Nektar Pulmonary Inhaler® (Nektar) efficiently removes powders from the packaging, breaks up the particles and creates an aerosol cloud suitable for deep lung delivery. It enables the aerosolized particles to be transported from the device to the deep lung during a patient's breath, reducing losses in the throat and upper airways. Compressed gas is used to aerosolize the powder. See AU4090599 and U.S. Pat. No. 5,740,794, the disclosure of each of which is incorporated herein by reference in their entireties.

NEXT DPI™ is a device featuring multidose capabilities, moisture protection, and dose counting. The device can be used regardless of orientation (upside down) and doses only when proper aspiratory flow is reached. See EP 1196146, U.S. Pat. No. 6,528,096, WO 2001/078693, and WO 2000/053158, the disclosure of each of which is incorporated herein by reference in their entireties.

Neohaler® is a capsule-based plastic breath-activated dry powder inhaler.

Oriel™ DPI is an active DPI that utilizes a piezoelectric membrane and nonlinear vibrations to aerosolize powder formulations. See International Patent Application Publication No. WO 2001/068169, the disclosure of which is incorporated herein by reference in its entirety.

RS01 monodose dry powder inhaler developed by Plastiape in Italy features a compact size and a simple and effective perforation system and is suited to both gelatin and HMPC capsules. The RS01 monodose DPI can be selected based on inspiratory resistances, with low, medium, high or ultra-high inspiratory resistances available.

Pressair™ is a plastic breath-activated dry powder inhaler.

Pulvinal® inhaler (Chiesi) is a breath-actuated multi-dose (100 doses) dry powder inhaler. The dry powder is stored in a reservoir which is transparent and clearly marked to indicate when the 100th dose has been delivered. See U.S. Pat. No. 5,351,683, the disclosure of which is incorporated herein by reference in its entirety.

The Rotohaler® (GlaxoSmithKline) is a single use device that utilizes capsules. See U.S. Pat. Nos. 5,673,686 and 5,881,721, the disclosure of each of which is incorporated herein by reference in their entireties.

Rexam DPI (Rexam Pharma) is a single dose, reusable device designed for use with capsules. See U.S. Pat. No. 5,651,359 and EP 0707862, the disclosure of each of which is incorporated herein by reference in their entireties.

S2 (Innovata PLC) is a re-useable or disposable single-dose DPI for the delivery of a dry powder composition in high concentrations. Its dispersion mechanism requires minimal patient effort to achieve excellent drug delivery to the patients' lungs. S2 is easy to use and has a passive engine so no battery or power source is required. See AU3320101, the disclosure of which is incorporated herein by reference in its entirety.

SkyeHaler® DPI (SkyePharma) is a multidose device containing up to 300 individual doses in a single-use, or replaceable cartridge. The device is powered by breath and requires no coordination between breathing and actuation. See U.S. Pat. No. 6,182,655 and WO 1997/020589, the disclosure of each of which is incorporated herein by reference in their entireties.

Taifun® DPI (LAB International) is a multiple-dose (up to 200) DPI device. It is breath actuated and flow rate independent. The device includes a unique moisture-balancing drug reservoir coupled with a volumetric dose metering system for consistent dosing. See U.S. Pat. No. 6,132,394, the disclosure of which is incorporated herein by reference in its entirety.

The TurboHaler® (AstraZeneca) is described in U.S. Pat. No. 5,983,893, the disclosure of which is incorporated herein by reference in its entirety. This DPI device is an inspiratory flow-driven, multi-dose dry-powder inhaler with a multi-dose reservoir that provides up to 200 doses of a dry powder composition and a dose range from a few micrograms to 0.5 mg.

The Twisthaler® (Schering-Plough) is a multiple dose device with a dose counting feature and is capable of 14-200 actuations. A dry powder composition is packaged in a cartridge that contains a desiccant. See U.S. Pat. No. 5,829,434, the disclosure of which is incorporated herein by reference in its entirety.

Ultrahaler® (Aventis) combines accurate dose metering and good dispersion. It is an easy-to-use, discrete, pocket-sized device with a numerical dose counter, dose taken indicator and a lock-out mechanism. The device is capable of delivering up to 20 mg of a dry powder composition. Ultrahaler® is described in U.S. Pat. No. 5,678,538 and WO 2004/026380, the disclosure of each of which is incorporated herein by reference in their entireties.

Xcelovair™ (Meridica/Pfizer) holds 60 pre-metered, hermetically sealed doses in the range of 5-20 mg. The device provides moisture protection under accelerated conditions of 40° C./75% RH. The dispersion system maximizes the fine particle fraction, delivering up to 50% fine particle mass.

A dry powder composition administered by one of the treatment methods provided herein is aerosolized via a DPI to provide aerosolized particles of the composition. In one embodiment, the aerosolized dry powder composition comprises particles with an MMAD of from about 1 μm to about 3 μm, as measured by NGI. In another embodiment, the aerosolized dry powder composition comprises particles with an MMAD of from about 1 μm to about 2 μm, as measured by NGI. In another embodiment, the aerosolized dry powder composition comprises particles with an MMAD of from about 1.5 μm to about 2 μm, as measured by NGI. In another embodiment, the aerosolized dry powder composition comprises particles with an MMAD of from about 1.5 μm to about 1.8 μm, as measured by NGI. In another embodiment, the aerosolized dry powder composition comprises particles with an MMAD of about 1.6 μm, as measured by NGI.

In one embodiment of the treatment methods disclosed herein, the aerosolized dry powder composition comprises particles with an FPF of from about 50% to about 75%, as measured by NGI. In another embodiment, the aerosolized dry powder composition comprises particles with an FPF of from about 60% to about 75%, as measured by NGI. In another embodiment, the aerosolized dry powder composition comprises particles with an FPF of from about 65% to about 75%, as measured by NGI. In another embodiment, the aerosolized dry powder composition comprises particles with an FPF of from about 68% to about 72%, as measured by NGI. In another embodiment, the aerosolized dry powder composition comprises particles with an FPF of about 71%, as measured by NGI.

In one embodiment of the disclosed treatment methods, the patient is administered the dry powder composition once daily. In a further embodiment, the dry powder composition administered according to the disclosed treatment methods comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1). In another embodiment of the disclosed treatment methods, the patient is administered the dry powder composition twice daily. In a further embodiment, the dry powder composition administered according to the disclosed treatment methods comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1). In still another embodiment of the disclosed treatment methods, the patient is administered the dry powder composition three or more times daily. In a further embodiment, the dry powder composition administered according to the disclosed treatment methods comprises a fumarate salt of bedaquiline, e.g., (αS, βR)-6-bromo-α-[2-(dimethylamino)ethyl]-2-methoxy-α-1-naphthalenyl-β-phenyl-3-quinolineethanol (2E)-2-butenedioate (1:1). In one embodiment, the administration is with food. In one embodiment, each administration comprises 1 to 5 doses (puffs) from a DPI, for example 1 dose (1 puff), 2 doses (2 puffs), 3 doses (3 puffs), 4 doses (4 puffs) or 5 doses (5 puffs). The DPI, in one embodiment, is small and transportable by the patient. In one embodiment, the dry powder inhaler is a single dose dry powder inhaler.

Yet another aspect of the disclosure relates to a system comprising (i) one of the dry powder compositions described herein and (ii) a dry powder inhaler (DPI). The DPI includes (a) a reservoir for holding the dry powder composition disclosed herein, and (b) a means for introducing the dry powder composition into the patient via inhalation. The reservoir in one embodiment, comprises the dry powder composition of the present disclosure in a capsule or in a blister pack. The material for the shell of a capsule can be gelatin, cellulose derivatives, starch, starch derivatives, chitosan, or synthetic plastics. The DPI may be a single dose or a multidose inhaler. In addition, the DPI may be pre-metered or device-metered. In one embodiment, the dry powder inhaler is a single dose dry powder inhaler. The system in one embodiment, is used for treating a mycobacterial lung disease, lung cancer, or a metastatic cancer to the lung in a patient, as described above.

### EXAMPLE

The present invention is further illustrated by reference to the following Examples. However, it should be noted that the Examples, like the embodiments described above, are illustrative and not to be construed as restricting the scope of the invention in any way.

### Example 1: Development and Characterization of Bedaquiline Fumarate Dry Powder Compositions

This example describes the development and characterization of dry powder compositions containing bedaquiline fumarate as the API. Bedaquiline fumarate dry powder for inhalation was prepared using various solvent systems and excipients and a Buchi B-290 spray dryer. We determined the morphology by scanning electron microscopy (SEM), particle size by using an Sympatec RODOS HELOS particle sizer, moisture content by using Karl Fischer titrimetry, as well as aerodynamic properties (e.g., MMAD and FPF) by NGI, crystalline or amorphous nature by X-ray diffraction (XRD), thermal phenomena by differential scanning calorimetry (DSC), surface composition by X-ray photoelectron spectroscopy (XPS), and residual solvent content by thermogravimetric analysis (TGA), of the dry powder compositions. This example shows that bedaquiline fumarate dry powder compositions containing 20% leucine or 20% trileucine as excipient displayed higher deposition of the excipient on particle surface (about 58% for leucine and about 44% for trileucine, respectively) compared to their respective proportions in the dry powder compositions. More importantly, bedaquiline fumarate dry powder composition containing 20% trileucine spray dried using an ethanol:water solvent system displayed stable wrinkled surface particle morphology, unimodal particle size distribution, desirable powder stability and efficient aerodynamic behavior over one year, with MMAD of 1.60 μm and FPF of 71% measured by NGI at t=0.

### Materials and Methods

### 1. Materials

Bedaquiline fumarate (Medchem Express)
Dipalmitoylphosphatidylcholine (DPPC) (Avanti Polar Lipids)
Leucine (Sigma)
Trileucine (Bachem)
Sodium chloride (Sigma)
1-propanol (Fisher Scientific)
Absolute ethanol (Fisher Bioreagents)
HyPure WFI quality water (GE Healthcare Life Sciences)

TABLE 2
Chemical structures of bedaquiline fumarate (API) and excipients

| Chemical | Structure |
|---|---|
| Bedaquiline fumarate | 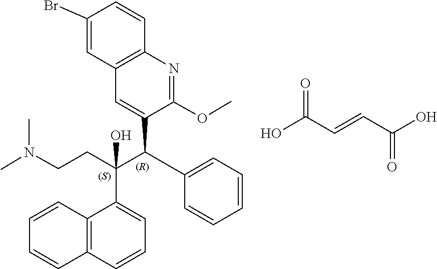 |
| DPPC | 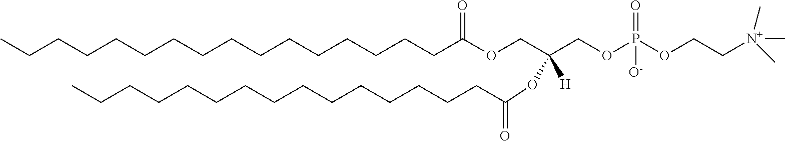 [SMILES: CCCCCCCCCCCCCCCCC(=O)OC[C@H](COP(=O)([O-])OCC[N+](C)(C)C)OC(=O)CCCCCCCCCCCCCCC] |
| Leucine | 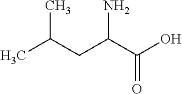 |
| Trileucine | 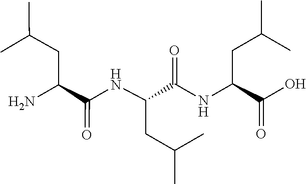 [SMILES: CC(C)C[C@H](NC(=O)[C@H](CC(C)C)NC(=O)[C@@H](N)CC(C)C)C(=O)O] |

### 2. Equipment

Buchi B-290 Spray Dryer with Inert Loop Condenser B-295, Dehumidifier B-296, Two-fluid nozzle ID 0.7 mm, and High-performance cyclonic separator (Buchi, Switzerland).
Scanning Electron Microscopy: Zeiss Sigma Field Emission SEM with Oxford INCA PentaFET×3 EDS system (Model 8100) with fully digital image collection, transfer and analysis.
Particle Size Diffraction: Sympatec Rodos Helos Laser Diffraction Particle Sizer (HELOS (H4234) & RODOS/M).
Karl Fischer titrator: Aquastar, AQV33, EMD.
XRD: Panalytical Xpert X-Ray Diffractometer.
NGI with pre-separator, stage cups, induction port and adapter appropriate for inhaler, MSP Corporation.
Differential Scanning calorimeter (DSC): TA Discovery DSC 250; Software: TA Trios.
Thermogravimetric Analysis (TGA): TA Discovery TGA 550; Software: TA Trios.
X-ray photoelectron spectroscopy (XPS): PHI Model 5701 LSCI.

### 3. Spray Drying

The Buchi B-290 spray dryer was assembled, according to the user manual. The dehumidifier (B-296) and inert loop (B-295) were attached to the spray dryer in order to operate the spray dryer in ‘closed’ mode. After assembly, the spray dryer and the dehumidifier were switched on. The aspirator was set to 100% and the spray gas (nitrogen) flow was set using the rotary valve. Once the pressure-drop threshold and oxygen concentration below 6% were reached (the respective signal lamps in the inert loop switch off), the desired inlet temperature was set and the heater was switched on. Once the desired inlet temperature was reached, the peristaltic pump was switched on and pure solvent was pumped in the spray dryer. After the outlet temperature stabilized, the feed tube was transferred to the flask containing the feed solution, which was spray dried. After the feed solution was completely sprayed, pure solvent was pumped for about 15 minutes. Once the feed tubes had been emptied out, the spray dryer was switched off as per the user manual.

The spray drying process parameters were varied as shown in Table 3. Table 4 is a summary of the exemplified bedaquiline fumarate dry powder compositions and their respective preparation process parameters. Spray dried powders were stored for stability testing at room temperature in a desiccator.

TABLE 3
Solvent systems and corresponding spray drying process parameters
for preparing bedaquiline fumarate dry powder compositions

| | Solvent system | |
|---|---|---|
| | 1-propanol:water | Ethanol:water |
| Parameters | (70:30 or 80:20) | (70:30 or 80:20) |
| Inlet Temperature (° C.) | 138-140 | 106-123 |
| Outlet Temperature (° C.) | 78-81 | 60-68 |
| Feed concentration (mg/mL) | 10-15.15 | 10 |
| Feed flow rate (mL/min) | 3.00 | 3.00 |
| Spraying gas flow (mm) | 30-44 | 39-52 |

TABLE 4
Exemplified bedaquiline fumarate dry powder compositions and their respective spray drying process parameters

| | | | | | | Process Parameters | | | | | | |
|---|---|---|---|---|---|---|---|---|---|---|---|---|
| | Composition Parameters | | | | | | | | | Feed | | |
| | | | | | Solid | | Feed | | | Flow | | |
| | | Composition | | | Conc | Batch | stock | Inlet | Outlet | Rate | | |
| Composition | Excipient | API/E1/ | Solvent | Solvent | (mg/ | size | volume | Temp | Temp | (mL/ | Aspira- | Q-flow |
| Identifier | E1, E2 | E2 | components | composition | mL) | (mg) | (mL) | (° C.) | (° C.) | min) | tion | (mm) |
| BDFDP1 | Trileucine | 90/10 | 1-propanol/water | 70/30 | 15.15 | 600 | 60 | 140 | 81 | 3 | 100 | 30 |
| BDFDP2 | Trileucine | 90/10 | 1-propanol/water | 70/30 | 15.15 | 600 | 60 | 140 | 78 | 3 | 100 | 44 |
| BDFDP3 | Trileucine | 90/10 | 1-propanol/water | 80/20 | 15.15 | 600 | 60 | 130 | 78 | 3 | 100 | 44 |
| BDFDP4 | Trileucine | 90/10 | 1-propanol/water | 70/30 | 10 | 600 | 60 | 138 | 78 | 3 | 100 | 44 |
| BDFPD5 | Trileucine | 80/20 | 1-propanol/water | 70/30 | 10 | 600 | 60 | 138 | 78 | 3 | 100 | 44 |
| BDFDP6 | Trileucine | 90/10 | ethanol/water | 70/30 | 10 | 600 | 60 | 120 | 68 | 3 | 100 | 44 |
| BDFDP8 | Trileucine | 80/20 | ethanol/water | 70/30 | 10 | 600 | 60 | 121 | 68 | 3 | 100 | 44 |
| BDFDP12 | NaCl | 90/10 | ethanol/water | 70/30 | 10 | 600 | 60 | 122 | 68 | 3 | 100 | 44 |
| BDFDP13 | Trileucine/NaCl | 80/15/5 | ethanol/water | 70/30 | 10 | 600 | 60 | 122 | 68 | 3 | 100 | 44 |
| BDFDP16 | DPPC | 90/10 | ethanol/water | 70/30 | 10 | 600 | 60 | 106 | 60 | 3 | 100 | 44 |
| BDFDP17 | Leucine | 80/20 | ethanol/water | 70/30 | 10 | 600 | 60 | 120 | 68 | 3 | 100 | 44 |
| BDFDP18 | NaCl | 98/2 | ethanol/water | 70/30 | 10 | 600 | 60 | 120 | 68 | 3 | 100 | 44 |
| BDFDP19 | NaCl | 98/2 | ethanol/water | 70/30 | 10 | 600 | 60 | 122 | 68 | 3 | 100 | 48 |
| BDFDP20 | NaCl | 98/2 | ethanol/water | 70/30 | 10 | 600 | 60 | 122 | 68 | 3 | 100 | 52 |

### 4. SEM Imaging

Sample powders were poured on a carbon tape and then coated with 20 nm gold (Au) using an Electron Microscopy Sciences (EMS150T ES) sputter coater. Field emission-scanning electron microscopy (FE-SEM) was used to observe the particle morphologies using a Zeiss-Sigma FE-SEM (Germany) with an operating voltage of 5 keV. The working distance was kept between 8 and 10 mm to obtain relatively high resolution.

### 5. Particle Size Distribution

Around 15 mg-20 mg of dry powder was put into the required glass tube. The Sympatec-HELOS-RODOS mode was used with the parameters shown in Table 5.

TABLE 5
Parameters for particle size distribution measurement
using the Sympatec HELOS RODOS

| | |
|---|---|
| Measuring range | R1: 0.1/0.18 . . . 35 μm |
| Trigger condition | Rodos standard trigger |
| Disperser | Rodos (RO-AS-15 mm) |
| Disperser type | RODOS/M |
| Injector | 4 mm with 0 cascade elements |
| Primary pressure | 1 bar |
| Vacuum | 22 mbar |

### 6. Karl Fischer Titrimetry

The moisture content in dry powder was analyzed using Karl Fischer with the materials and operating parameters shown in Table 6. The equipment included an Aquastar AQV33 Karl Fischer Titrator with 5 mL burette installed, and an associated analytical balance capable of weighing up to 4-decimal places with an interface capable of being connected to the Aquaster Titrator. Approximately 30 mg of sample was weighed out and transferred to the titration vessel.

TABLE 6
Parameters for Karl Fischer Titrimetry

| | |
|---|---|
| Titrant | Aquaster Combi Titrant 2 |
| Standard | Water Standard 1% NIST |
| Solvent | Aquaster Combi Coulomat fritness |
| Drift | <50 μg/min |
| Potential | 80-100 |
| Mix Time | 300 s |
| Termination | Relative Drift |

### 7. Differential Scanning Calorimetry (DSC)

An empty TA Tzero pan was hermetically sealed using TA Tzero Hermetic Lid and weighed. This pan was used as the Reference pan. 5-10 mg of dry powder was added to another TA Tzero Pan (identified as Sample pan). The pan was then hermetically sealed using TA Tzero Hermetic Lid. Both of the pans were placed in the autosampler of the TA Discovery DSC 250 and the sample holder number was noted.

Table 7 shows the parameters used for the DSC run. The DSC thermogram obtained at the end of the run was then analyzed.

TABLE 7
DSC run parameters

| No. | Description |
|---|---|
| 1 | Equilibrate 20.00° C. |
| 2 | Modulate Temperature 1.00° C. for 60.0 s |
| 3 | Isothermal 5.0 min |
| 4 | Ramp 10.00° C./min to 300.00° C. |

### 8. Thermogravimetric Analysis (TGA)

An empty TA Tzero pan was placed on the platinum TGA pan holder and tared. After taring, the TZero pan was transferred to a humidity-controlled glove box, where approximately 5-10 mg of the dry powder was added to it. The filled TZero pan was then transferred back to the platinum pan holder. The TGA run was then initiated. The parameters of the run were: Ramp 20° C./min to 160° C. The weight change was measured using the TRIOS software.

### 9. X-Ray Diffraction (XRD) Analysis

Dry powder was packed in the zero-background sample holder and then XRD was employed for assessment of the structural characteristics using a PANalytical (Netherlands) X′Pert Diffractometer at 45 kV and 40 mA with Cu Kα (2=1.540598 Å) radiation. The scanning range was between 4° and 50° degrees (20) with a time per step of 97.92 seconds and a step size of 0.0131°.

### 10. Analysis of Aerodynamic Properties Using the NGI

NGI was assembled with pre-separator, induction port adapter and filter assembly and placed into the environmental chamber equilibrated to 35° C. and 15% RH. The flow rate was adjusted to 60 L/min. Approximately 10 mg dry powder composition was added to a gel capsule and the capsule was loaded in the inhaler. The pins of the inhaler were pressed to pierce the capsule, the loaded inhaler was inserted into the induction port via adapter, and the valve into the NGI was opened for 4 seconds. The valve was then closed, and the pump was turned off. The weight of the total sample aerosolized was determined by calculating the difference between the weights of the filled capsule and the aerosolized capsule. The weight of the powder residue inside the inhaler was calculated by the difference between the weights of the empty inhaler after aerosolization and the empty inhaler before aerosolization. The NGI was then disassembled and samples were collected from the induction port, adapter, pre-separator, stages, filter, inhaler and capsule. The samples were diluted and analyzed using HPLC.

### 11. Bulk and Tap Density Measurement

An empty micro glass tube was weighed along with its lid. Spray dried powder was added to the microtube to fill roughly ⅔^{rd} tube without compacting and the lid was tightly closed. The height of the powder inside the microtube was marked on the tube and the microtube was weighed. The microtube was placed inside the graduated cylinder of the tap density tester and tapped for 10 minutes. After tapping, the microtube was taken out gently from the graduated cylinder and the powder height after tapping was marked. The dry powder from the microtube was discarded and the microtube was rinsed with water and ethanol to remove any remaining powder and air dried. The rinsed and dried microtube was reweighed. Using a dropper, water was added to the microtube up to the tapped powder marking, the lid was tightly closed and the microtube was weighed. Next, more water was added to the microtube up to the bulk powder marking, the lid was tightly closed and the microtube was weighed. The tap and bulk densities were calculated.

### 12. X-Ray Photoelectron Spectroscopy (XPS)

XPS was used to determine the proportions of the API and excipient on the surface of the spray dried particles of dry powder composition BDFDP8 containing 20% trileucine and dry powder composition BDFDP17 containing 20% leucine, with the analytical parameters shown in Table 8.

TABLE 8
XPS parameters

| | |
|---|---|
| Instrument | PHI 5701 LSCI |
| X-ray source | Monochromated Alk_{α} 1486.6eV |
| Acceptance Angle | +7° |
| Take-off angle | 50° |
| Analysis area | 2.0 mm × 0 .8 mm |
| Charge Correction | C1s 284.8eV (C—C, C—H) |

### Results

### 1. Powders Spray Dried Using 1-Propanol:Water Solvent System

1-propanol:water solvent system was initially used to spray dry bedaquiline fumarate powders.

### 1.1. Yield

Yields between 80-90% were achieved for bedaquiline fumarate dry powder compositions containing trileucine.

### 1.2. Residual Moisture Content

All dry powder compositions displayed residual moisture content of less than 1% as determined by Karl Fisher titrimetry.

### 1.3. SEM Analysis

Dry powder compositions generally displayed a spherical morphology. Dry powder compositions containing 10-20% trileucine, e.g., dry powder composition BDFDP4 containing 10% trileucine, showed presence of small round protrusions on the surface, indicative of possible surface deposition of trileucine. Increasing the trileucine proportion from 10% in dry powder composition BDFDP4 to 20% in dry powder composition BDFDP5 resulted in a slight wrinkled surface.

### 1.4. Geometric Particle Size Distribution

Dry powder composition BDFDP4 containing 10% trileucine exhibited a unimodal particle size distribution preceded by a small plateau and mean particle size of 2.81 to 3.31 μm. Increasing the proportion of trileucine to 20% in dry powder composition BDFDP5 did not change the particle size distribution or mean particle size (2.72 μm). Increasing the spraying gas flow and reducing the feed concentration led to a slight decrease in mean geometric size.

### 1.5. Aerodynamic Properties

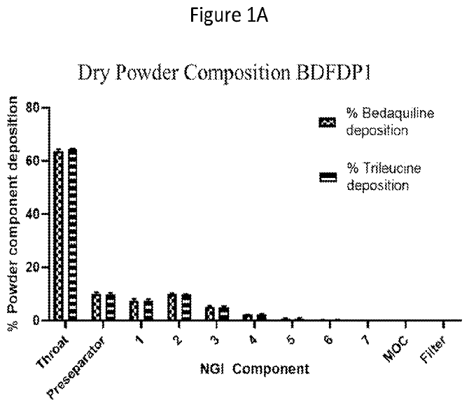

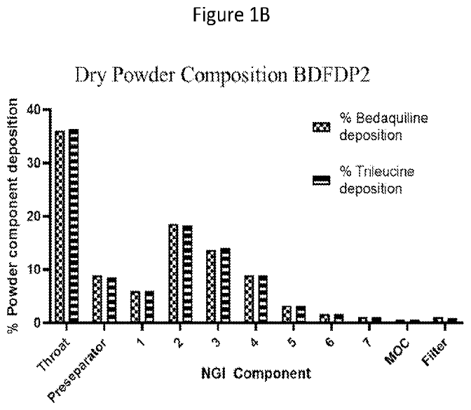

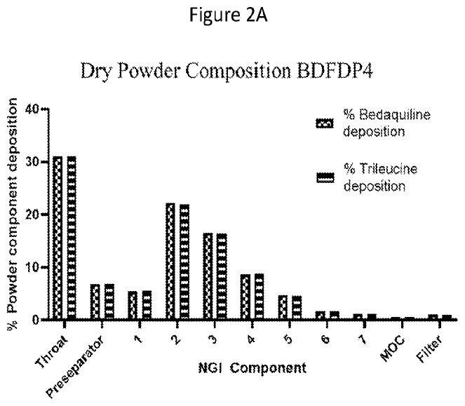

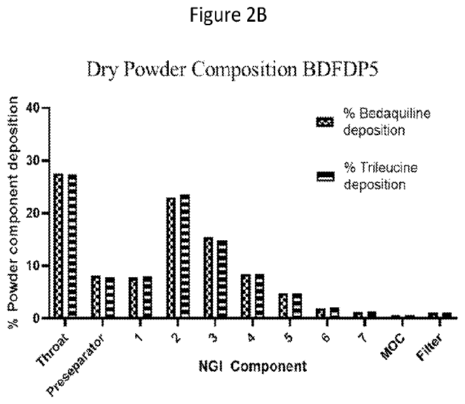

Aerodynamic properties of bedaquiline fumarate dry powder compositions containing 10% or 20% trileucine and spray dried using 1-propanol:water solvent system are shown in Table 9. Increasing the spray gas flow improved aerodynamic performance of the dry powder, as can be seen from the lower throat and preseparator deposition for dry powder composition BDFDP2 compared to dry powder composition BDFDP1 (Tables 4 and 9; FIGS. 1A and 1B). Increasing the proportion of trileucine from 10% in dry powder composition BDFDP4 to 20% in dry powder composition BDFDP5 did not enhance the aerodynamic performance of the dry powder (Table 9; FIGS. 2A and 2B).

TABLE 9
Aerodynamic properties of bedaquiline fumarate/trileucine dry powder
compositions spray dried using 1-propanol:water solvent system

| | | | | Throat + | |
|---|---|---|---|---|---|
| | | | | Presep | |
| | | | | Deposition | |
| | | | | Based on | |
| | | Composition | | Emitted | FPF |
| Composition | | API/Excipient | MMAD | Dose | (<5 μm) |
| Identifier | Excipient | w/w | μm | % | % |
| BDFDP1 | Trileucine | 90/10 | 6.85 | 73.83 | 9.8 |
| BDFDP2 | Trileucine | 90/10 | 3.57 | 45.00 | 34.3 |
| BDFDP3 | Trileucine | 90/10 | 4.15 | 41.30 | 33.9 |
| BDFDP4 | Trileucine | 90/10 | 3.44 | 37.90 | 39.9 |
| BDFDP5 | Trileucine | 80/20 | 3.78 | 35.70 | 38.8 |

### 2. Powders Spray Dried Using Ethanol:Water Solvent System

Since bedaquiline fumarate dry powder compositions spray-dried using 1-propanol:water displayed a low FPF (<40%), the solvent system was changed to ethanol: water. It was hypothesized that ethanol, due to its lower boiling point, would begin to evaporate before the water during spray drying and result in a change in the composition of the solvent system. This could lead to the excipient precipitating from solution and depositing on the surface of the drying droplet, leading to its presence on the surface of the dried particle. Ethanol's lower boiling point would also allow for lower inlet and outlet temperatures during spray drying, reducing the possibility of thermal degradation or phase transitions of the dried powder. Amino acids leucine and trileucine were used as excipients for their effects on dispersibility and aerosolization properties of spray dried powders. Other excipients such as NaCl and DPPC were also investigated.

### 2.1. Yield

Spray dried powders displayed yields between 60-90%, depending on the excipients. The lowest yield was observed for the dry powder composition containing 2% sodium chloride at 59.47%, while the dry powder composition containing 20% leucine had a yield of 90.17%.

### 2.2. SEM Analysis

The morphology of dry powders was affected by the choice of excipients. Dry powder composition BDFDP8 with 20% trileucine displayed particles with wrinkled surface and slight particle breakage. Dry powder composition BDFDP16 with 10% DPPC and dry powder composition BDFDP17 with 20% leucine displayed collapsed particle morphology with no particle breakage. Dry powder composition BDFDP12 with 10% sodium chloride displayed a porous particle surface. Dry powder composition BDFDP17 with 20% leucine displayed morphological stability up to 3.5 months of storage in a desiccator at room temperature. In dry powder compositions containing trileucine, increasing the proportion of trileucine from 10% in dry powder composition BDFDP6 to 20% in dry powder composition BDFDP8 did not affect particle morphology. Dry powder composition BDFDP8 containing 20% trileucine was morphologically stable after 2 months of storage in a desiccator at room temperature. Furthermore, both dry powder composition BDFDP8 containing 20% trileucine and dry powder composition BDFDP17 containing 20% leucine were morphologically unchanged by SEM over one year of storage in a desiccator at room temperature.

### 2.3. Geometric Particle Size Distribution

All dry powders had mean geometric particle sizes between 2 and 4 μm. Dry powders containing 20% leucine (dry powder composition BDFDP17), 10% trileucine (dry powder composition BDFDP6), or 20% trileucine (dry powder composition BDFDP8) exhibited unimodal particle size distribution. Dry powders with trileucine displayed a broader distribution peak compared to dry powder with leucine. Dry powder composition BDFDP16 containing 10% DPPC and dry powder composition BDFDP12 containing 10% sodium chloride displayed bimodal-like distribution.

Dry powders with 2% sodium chloride (dry powder compositions BDFDP18, BDFDP19, and BDFDP20) were prepared to study the effect of sodium chloride on reducing powder static. Dry powder compositions BDFDP18, BDFDP19, and BDFDP20 were prepared at low, medium, and high drying gas rates, respectively (Table 4). Particle size distribution was generally unimodal and depended on the spraying gas rate.

Dry powder composition BDFDP8 with 20% trileucine did not display a significant change in particle size distribution and particle size after storing the dry powder in a desiccator at room temperature for 2 months.

Particle size distribution of both dry powder composition BDFDP8 containing 20% trileucine and dry powder composition BDFDP17 containing 20% leucine was stable over one year of storage in a desiccator at room temperature, as shown by their respective median particle diameters (d₅₀) before the storage (t=0), at 6 months of storage (t=6 months), and at 12 months of storage (t=12 months) (Table 10).

TABLE 10
Median particle diameters (d₅₀) of bedaquiline fumarate
dry powder compositions BDFDP8 and BDFDP17
at t = 0, t = 6 months, and t = 12 months

| | d₅₀ (μm) | |
|---|---|---|
| | BDFDP8 | BDFDP17 |
| t = 0 | 2.02 | 2.20 |
| t = 6 months | 1.92 | 2.62 |
| t = 12 months | 1.95 | 2.51 |

### 2.4. Aerodynamic Properties

Aerodynamic properties of spray dried powders were analyzed using NGI, with the data shown in Table 11. Dry powder composition BDFDP8 containing 20% trileucine displayed the most suitable aerodynamic properties with the lowest MMAD of 1.60 μm and highest FPF of 71%.

TABLE 11
Aerodynamic properties of bedaquiline fumarate
spray dried powders at t = 0

| | | | | Throat + | |
|---|---|---|---|---|---|
| | | | | Presep | |
| | | | | Deposition | |
| | | | Geometric | Based on | |
| | | | Standard | Emitted | FPF |
| Composition | | MMAD | Deviation | Dose | (<5 μm) |
| Identifier | Excipient | μm | (GSD) | % | % |
| BDFDP6 | 10% Trileucine | 2.90 | 2.88 | 13.90 | 60.10 |
| BDFDP8 | 20% Trileucine | 1.60 | 3.57 | 12.90 | 71.00 |
| BDFDP12 | 10% NaCl | 3.19 | 2.35 | 21.70 | 55.00 |
| BDFDP13 | 15% Trileucine | 3.01 | 3.27 | 20.20 | 53.20 |
| | and 5% NaCl | | | | |
| BDFDP16 | 10% DPPC | 2.53 | 2.22 | 38.92 | 49.14 |
| BDFDP17 | 20% Leucine | 3.51 | 3.08 | 33.90 | 41.30 |
| BDFDP18 | 2% NaCl | 3.02 | 2.48 | 32.40 | 48.00 |

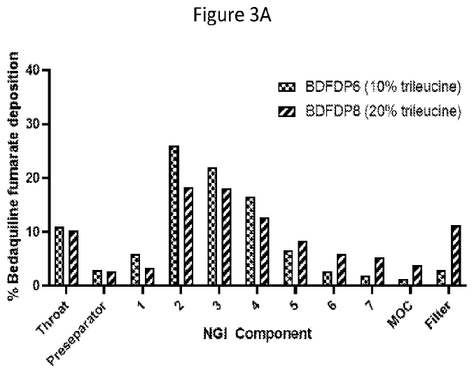

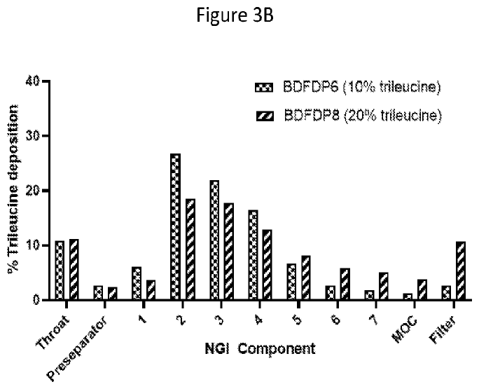

For dry powder composition BDFDP6 containing 10% trileucine and dry powder composition BDFDP8 containing 20% trileucine, deposition of both bedaquiline fumarate and trileucine in the NGI was similar, indicating that both these components were uniformly distributed in the dry powder particles (FIGS. 3A and 3B). Dry powder composition BDFDP6 displayed an FPF of 60.10% and MMAD of 2.90 μm. Increasing the proportion of trileucine to 20% in dry powder composition BDFDP8 significantly enhanced the FPF to 71% and reduced the MMAD to 1.60 μm (Table 11).

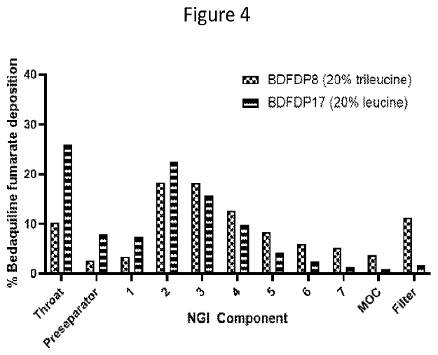

Dry powder composition BDFDP17 containing 20% leucine displayed higher deposition of the bedaquiline fumarate component in the throat and preseparator regions of the NGI, while higher deposition of the bedaquiline fumarate component in stages 3 onwards was observed for dry powder composition BDFDP8 containing 20% trileucine (FIG. 4). Dry powder composition BDFDP17 displayed inferior aerodynamic properties (FPF of 41.30% and MMAD of 3.51 μm) compared to dry powder composition BDFDP8 containing 20% trileucine (Table 11).

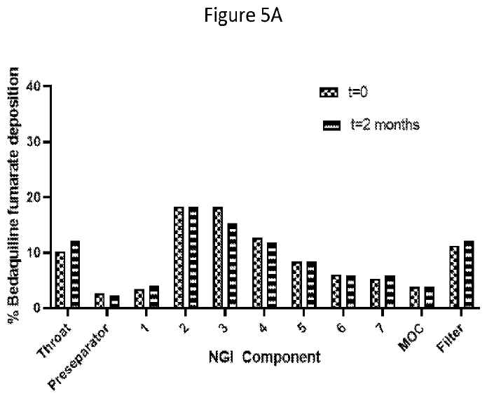

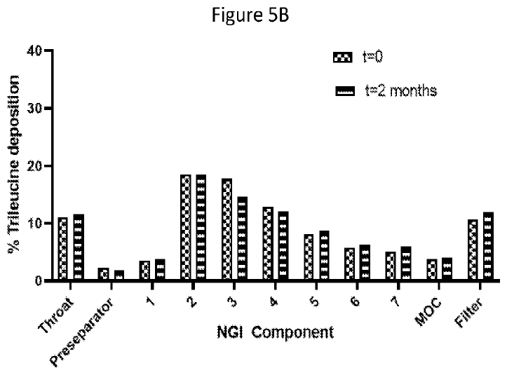

Dry powder composition BDFDP8 containing 20% trileucine displayed similar deposition upon aerosolization in the NGI after storage in a desiccator at room temperature for 2 months. Both the bedaquiline fumarate and trileucine components of the dry powder displayed similar deposition at both times, indicating that the uniform deposition of the components in the dry powder particles remained intact after 2 months of storage (FIGS. 5A and 5B).

Dry powder composition BDFDP8 also displayed similar aerodynamic properties after 2 months of storage in a desiccator at room temperature, thus highlighting the aerodynamic stability of the dry powder (Table 12).

TABLE 12
Aerodynamic properties of dry powder composition BDFDP8
containing 20% trileucine at t = 0 and t = 2 months

| | | | | Throat + | |
|---|---|---|---|---|---|
| | | | | Preseperator | FPF |
| Dry powder | | MMAD | | Deposition | (<5 μm) |
| component | | μm | GSD | % | % |
| Bedaquiline | t = 0 | 1.60 | 3.57 | 12.90 | 71.00 |
| fumarate | t = 2 months | 1.54 | 3.87 | 14.50 | 69.10 |
| Trileucine | t = 0 | 1.63 | 3.59 | 13.50 | 70.00 |
| | t = 2 months | 1.52 | 3.81 | 13.50 | 70.40 |

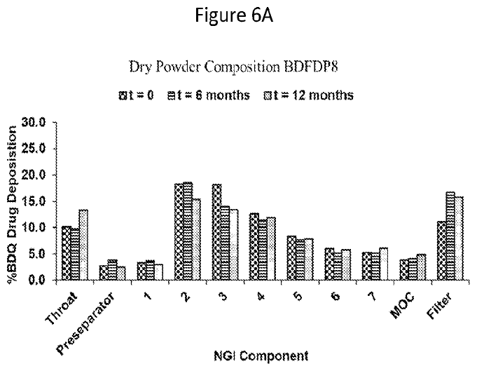

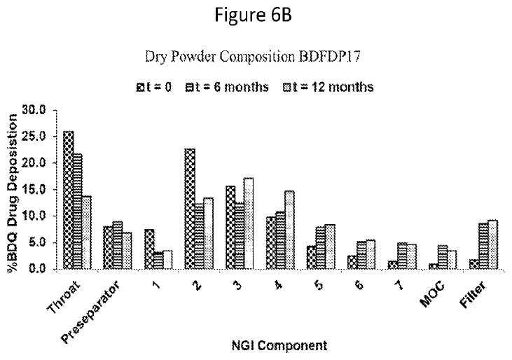

Dry powder composition BDFDP8 containing 20% trileucine and dry powder composition BDFDP17 containing 20% leucine each were stored in a desiccator at room temperature, and deposition of the bedaquiline fumarate component of each composition in NGI stages based on emitted dose was determined before the storage (t-0), at 6 months of storage (t=6 months), and at 12 months of storage (t=12 months) (FIGS. 6A and 6B). Dry powder composition BDFDP8 displayed stable deposition pattern over the one-year storage period, while dry powder composition BDFDP17 displayed changes in powder deposition. Dry powder composition BDFDP8 also showed stable aerodynamic properties measured by NGI over one year of storage, in contrast to dry powder composition BDFDP17 (Tables 13A and 13B). Notably, dry powder composition BDFDP8 displayed no change in fine particle fraction (FPF). By contrast, dry powder composition BDFDP17 displayed a sharp increase in FPF from 41% to 65% over one year, potentially making dose determination unpredictable.

TABLE 13A
Aerodynamic properties of dry powder composition BDFDP8
containing 20% trileucine at t = 0, t = 6 months, and t = 12 months

| | | | | Throat + | |
|---|---|---|---|---|---|
| | | | | Preseperator | |
| | | | | Deposition | |
| | | | | Based on | |
| | | | | Emitted | FPF |
| Dry powder | | MMAD | | Dose | (< 5 μm) |
| component | | μm | GSD | % | % |
| Bedaquiline | t = 0 | 1.60 | 3.57 | 12.9 | 71.0 |
| fumarate | t = 6 months | 1.36 | 4.26 | 13.6 | 70.5 |
| | t = 12 months | 1.23 | 4.04 | 15.8 | 70.9 |

TABLE 13B
Aerodynamic properties of dry powder composition BDFDP17
containing 20% leucine at t = 0, t = 6 months, and t = 12 months

| | | | | Throat + | |
|---|---|---|---|---|---|
| | | | | Preseperator | |
| | | | | Deposition | |
| | | | | Based on | FPF |
| Dry powder | | MMAD | | Emitted Dose | (<5 μm) |
| component | | μm | GSD | % | % |
| Bedaquiline | t = 0 | 3.51 | 3.08 | 33.9 | 41.3 |
| fumarate | t = 6 months | 1.45 | 3.70 | 30.6 | 57.5 |
| | t = 12 months | 1.60 | 3.41 | 20.5 | 65.5 |

Bulk density and tap density were compared between dry powder composition BDFDP8 containing 20% trileucine and dry powder composition BDFDP17 containing 20% leucine (Table 14).

TABLE 14
Bulk density and tap density of dry powder
compositions BDFDP8 and BDFDP17

| | | Bulk | Tap |
|---|---|---|---|
| Composition | | density | density |
| Identifier | Excipient | (g/cm³) | (g/cm³) |
| BDFDP8 | 20% Trileucine | 0.241 | 0.441 |
| BDFDP17 | 20% Leucine | 0.251 | 0.430 |

### 2.5. XRD Analysis

XRD analysis revealed that dry powder composition BDFDP8 containing 20% trileucine was amorphous in nature, while dry powder composition BDFDP17 containing 20% leucine was crystalline.

### 2.6. XPS Analysis

XPS analysis revealed that dry powder composition BDFDP8 containing 20% trileucine displayed about 44% trileucine deposition on the particle surface, while dry powder composition BDFDP17 containing 20% leucine displayed about 58% leucine deposition on the particle surface. Both excipients displayed higher deposition on the particle surface compared to their proportion in the spray dried powder, highlighting the high surface activity of amino acids.

### 2.7. DSC Analysis

Both dry powder composition BDFDP8 containing 20% trileucine and dry powder composition BDFDP17 containing 20% leucine exhibited no significant changes in thermal profiles as determined by DSC, following storage in a desiccator at room temperature for 6 months (t=6 months) or 12 months (t=12 months), as compared to their respective thermal profiles before the storage (t=0).

### 2.8 TGA Analysis

Both dry powder composition BDFDP8 containing 20% trileucine and dry powder composition BDFDP17 containing 20% leucine displayed a reduction in residual solvent content over one-year storage period, possibly due to the presence of desiccator.

In summary, the data of this example demonstrate that dry powder composition BDFDP8 with 20% trileucine spray dried from an ethanol:water (70:30) solvent system displayed wrinkled surface, unimodal particle size distribution (geometric particle size between 2 and 2.5 μm) and good aerodynamic properties (MMAD<2 μm and FPF of 71% using the NGI). Dry powder composition BDFDP8 also displayed no changes in thermal profiles, morphology, and particle size distribution over one year. Additionally, the data indicate that dry powders containing 20% amino acids (leucine or trileucine) displayed greater excipient deposition on particle surface compared to their proportion in the dry powder.

### Example 2: Single Dose Pharmacokinetic Evaluation of Inhaled Bedaquiline Fumarate Dry Powders in Rats

In this example, the lung and plasma pharmacokinetics (PK) of bedaquiline fumarate dry powder compositions BDFDP8 containing 20% trileucine and BDFDP17 containing 20% leucine administered via nose-only inhalation using the Vilnius Aerosol Generator (VAG) dry powder disperser, as well as the lung and plasma PK of a bedaquiline fumarate solution administered orally, in healthy male Sprague Dawley rats were determined and compared.

### Materials and Methods

Sprague Dawley male rats weighing 175-200 g were purchased from Envigo. Two cohorts of male Sprague Dawley rats each (n=11 per cohort) were pre-weighed and exposed to a single inhaled dose of bedaquiline fumarate dry powder composition BDFDP8 containing 20% trileucine or dry powder composition BDFDP17 containing 20% leucine, using nose-only inhalation via the VAG. An additional cohort of 21 animals were pre-weighed and subjected to oral dosing of bedaquiline fumarate via gavage.

The inhaled dosing cohorts were loaded into a 12-port nose-only inhalation system (CH Technologies, Westwood, NJ) for inhaled dosing. An aerosol sampling filter was placed on one of the available nose-only inhalation ports for determination of the dose delivered to the nose, and a Marple impactor was placed on the remaining inhalation port to determine the aerodynamic particle size distribution (APSD) of aerosolized dry powder composition. Approximately 265 mg of the powder was loaded to the generator for each of the cohorts. The VAG was set to a 1 V output and fed with dry compressed air at a flow rate equal to 8 L/min. Animals were dosed with the bedaquiline fumarate dry powder for approximately 30 min.

For the oral dosing cohort, 1 mL syringes were connected to disposable feeding tubes and a bedaquiline fumarate oral solution containing 2.5 mg/mL bedaquiline fumarate was pulled into connected unit until 0.1 mL mark was reached. The feeding tube was gently inserted into the esophagus until the stomach was reached. Approximately 1 mL of bedaquiline fumarate oral solution (approximately 4 mL/kg body weight) was administered per animal from the pre-loaded syringe/feeding tube and the feeding tube was gently removed.

Plasma and whole lungs were collected post-dose at the following timepoints: immediate post dose (IPD, approximately 30 min), at 4 h, day 1, day 3 and day 7. Blood was taken, centrifuged at 14,000 rpm for 10 minutes and transferred to pre-labeled tubes. Lung weights were recorded at necropsy. Lung samples were taken at the same time-points for determining lung concentrations of bedaquiline fumarate. Plasma and lung samples were stored at −20° C. The lung and plasma levels of bedaquiline fumarate were determined by liquid chromatography tandem mass spectrometry (LC-MS/MS).

The concentration of bedaquiline fumarate on the sampling filter was quantified using HPLC with UV Detector set to 280 nm. The dose delivered to the rats at the nose was calculated based on the following equation:

$$Dose ( mg / kg ) = \frac{\begin{matrix}respiratory minute volume ( RMV , L / min ) \times \\ aerosol concentration ( mg / L ) \times Dose Time ( min )\end{matrix}}{Body Weight ( kg )}$$

Analysis of the lung and plasma PK was performed using the open-source software add-in PKSolver 2.0 for Microsoft Excel. The data were applied to a noncompartmental analysis after extravascular dosing input and fit to a linear up/log down model (the linear trapezoidal method).

### Results

For the cohort treated with a single inhaled dose of bedaquiline fumarate dry powder composition BDFDP8 containing 20% trileucine, the average amount of powder dispensed was 245±13 mg, the average dose delivered at the nose as determined by the aerosol sampling filter was 7.18±0.09 mg/kg, and the average IPD pulmonary dose was 0.29±0.03 mg/kg (approximately 4% of the nose dose).

For the cohort treated with a single inhaled dose of bedaquiline fumarate dry powder composition BDFDP17 containing 20% leucine, the average amount of powder dispensed was 254±1 mg, the average dose delivered at the nose was 8.71±0.75 mg/kg, and the average IPD pulmonary dose was 0.21±0.05 mg/kg (approximately 2.5% of the nose dose).

For the oral cohort, a dose of 10 mg/kg was delivered orally.

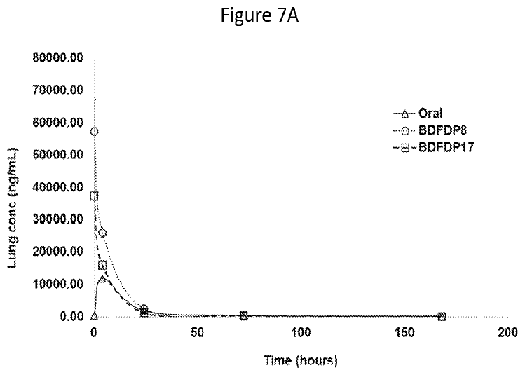

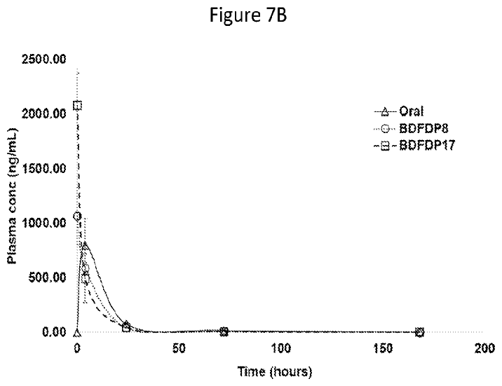

The lung and plasma levels of bedaquiline fumarate after inhalation of bedaquiline fumarate dry powder composition BDFDP8 or BDFDP17, or oral administration of the bedaquiline fumarate solution over the course of 7 days (168 hours) are shown in FIGS. 7A and 7B, respectively.

With inhaled dry powder composition BDFDP8, the average IPD (0.5 h) lung concentration of bedaquiline fumarate was 50.16 μg/g, which was also the maximum concentration (Cₘₐₓ). This was reduced to 2.97 μg/g by Day 1, demonstrating a 94% reduction over this time period (FIG. 7A). Plasma Cₘₐₓ was 1.05 μg/mL at 0.5 h after dose (FIG. 7B). This was 2.1% of lung Cₘₐₓ at the same timepoint, which shows that the drug yields markedly low systemic levels when given in a single dose by inhalation. The bedaquiline fumarate lung AUC₀₋₇ Day was calculated to be 22.14 μg*day/g and the plasma AUC 0-7 Day was 0.54 μg*day/mL, yielding an AUC ratio of approximately 41:1 lung:plasma. Table 15A is a summary of the PK parameters calculated for inhaled dry powder composition BDFDP8 containing 20% trileucine.

TABLE 15A
PK parameters for inhaled dry powder composition BDFDP8 containing 20% trileucine

| VAG | | | | Cₘₐₓ | | Cₘᵢₙ | | | |
|---|---|---|---|---|---|---|---|---|---|
| Run | VAG | Nose | | (μg/g) | | (μg/g) | | | AUC_{0-7 Day} |
| Time | Voltage | Dose | | or | Tₘₐₓ | or | Tₘᵢₙ | t_{1/2} | (μg*day/g) or |
| (min) | (V) | (mg/kg) | Tissue | (μg/mL) | (Day) | (μg/mL) | (Day) | (Day) | (μg*day/mL) |
| 30 | 1 | 7.18 | Lung | 50.16 | 0.021 | 0.10 | 7 | 0.82 | 22.14 |
| | | | Plasma | 1.05 | 0.021 | 0.00 | 7 | 1.70 | 0.54 |

With inhaled dry powder composition BDFDP17, the average IPD (0.5 h) lung concentration of bedaquiline fumarate was 34.53 μg/g, which was also the Cₘₐₓ. This was reduced to 1.57 μg/g by Day 1, demonstrating a 96% reduction over this time period (FIG. 7A). Plasma Cₘₐₓ was 2.01 μg/mL at 0.5 h after dose (FIG. 7B). This was 5.8% of lung Cₘₐₓ at the same timepoint, which shows that the drug yields markedly low systemic levels when given in a single dose by inhalation. The bedaquiline fumarate lung AUC_{0-7 Day} was calculated to be 13.72 μg*day/g and the plasma AUC_{0-7 Day} was 0.58 μg*day/mL, yielding an AUC ratio of approximately 24:1 lung: plasma. Table 15B is a summary of the PK parameters calculated for inhaled dry powder composition BDFDP17 containing 20% leucine.

TABLE 15B
PK parameters for inhaled dry powder composition BDFDP17 containing 20% leucine

| VAG | | | | | | Cₘᵢₙ | | | |
|---|---|---|---|---|---|---|---|---|---|
| Run | VAG | Nose | | Cₘₐₓ | | (μg/g) | | | AUC_{0-7 Day} |
| Time | Voltage | Dose | | (μg/g) | Tₘₐₓ | or | Tₘᵢₙ | t_{1/2} | (μg*day/g) or |
| (min) | (V) | (mg/kg) | Tissue | or | (Day) | (μg/mL) | (Day) | (Day) | (μg*day/mL) |
| 30 | 1 | 8.71 | Lung | 34.53 | 0.021 | 0.10 | 7 | 1.59 | 13.72 |
| | | | Plasma | 2.01 | 0.021 | 0.00 | 7 | 1.72 | 0.58 |

With the orally administered bedaquiline fumarate solution, the average IPD (0.5 h) lung concentration of bedaquiline fumarate was 0.38 μg/g. Cₘₐₓ of 9.77 μg/g was observed at 4 h post dose. This was reduced to 1.61 μg/g by Day 1, displaying an 84% reduction over this time period (FIG. 7A). Plasma Cₘₐₓ was 0.65 μg/mL at 4 h after dose (FIG. 7B). This was 6.8% of lung Cₘₐₓ at the same timepoint, which shows that the drug yields markedly low systemic levels when given in a single oral dose. The bedaquiline fumarate lung AUC 0-7 Day was calculated to be 8.32 μg*day/g and the plasma AUC_{0-7 Day} was 0.47 μg*day/mL, yielding an AUC ratio of approximately 18:1 lung: plasma. Table 15C is a summary of the PK parameters calculated for the bedaquiline fumarate oral formulation.

TABLE 15C
PK parameters for bedaquiline fumarate oral formulation

| | | Cₘₐₓ | | Cₘᵢₙ | | | AUC_{0-7 Day} |
|---|---|---|---|---|---|---|---|
| | | (μg/g) | | (μg/g) | | | (μg*day/g) |
| Dose | | or | Tₘₐₓ | or | Tₘᵢₙ | t_{1/2} | or |
| (mg/kg) | Tissue | (μg/mL) | (Day) | (μg/mL) | (Day) | (Day) | (μg*day/mL) |
| 10 | Lung | 9.77 | 0.17 | 0.12 | 7 | 1.70 | 8.32 |
| | Plasma | 0.65 | 0.17 | 0.00 | 7 | 1.52 | 0.47 |

In summary, bedaquiline fumarate lung Cₘₐₓ was higher in the inhaled BDFDP8 dry powder (containing 20% trileucine) cohort (50.16 μg/g) than in the inhaled BDFDP17 dry powder (containing 20% leucine) cohort (34.53 μg/g) at 30 min. Bedaquiline fumarate lung concentration was reduced to approximately 0.10 μg/g by Day 7 for both dry powder compositions BDFDP8 and BDFDP17 following inhaled administration. The lung bedaquiline fumarate t_{1/2} with inhaled BDFDP17 dry powder was double (1.59 days) that with inhaled BDFDP8 dry powder (0.82 days). Peak plasma levels of bedaquiline fumarate were also seen at 30 min after dosing with either of the dry powders, and were twice as high with inhaled BDFDP17 dry powder (2.005 μg/mL) as compared to inhaled BDFDP8 dry powder (1.046 μg/mL). The lung AUCs over the 7-day timecourse for inhaled BDFDP8 dry powder and inhaled BDFDP17 dry powder were 22.14 μg*d/g and 13.72 μg*d/g, respectively, indicating a higher bedaquiline fumarate lung exposure with BDFDP8 dry powder containing 20% trileucine. The plasma AUCs over the same timecourse for both the dry powders were similar (0.54 μg*d/mL for BDFDP8 and 0.58 μg*d/mL for BDFDP17), indicating similar plasma bedaquiline fumarate exposure irrespective of the choice of amino acid in the dry powder.

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

Patents, patent applications, patent application publications, journal articles and protocols referenced herein are incorporated by reference in their entireties, for all purposes.

1. A dry powder composition comprising:
(a) from about 65 wt % to about 95 wt % of bedaquiline, or a pharmaceutically acceptable salt thereof; and
(b) the balance being trileucine;
wherein the entirety of (a) and (b) is 100 wt %.

2. The dry powder composition of claim 1, wherein bedaquiline, or a pharmaceutically acceptable salt thereof is present at from about 68 wt % to about 95 wt % of the total weight of the dry powder composition.

3. The dry powder composition of claim 1, wherein bedaquiline, or a pharmaceutically acceptable salt thereof is present at from about 70 wt % to about 95 wt % of the total weight of the dry powder composition.

4. The dry powder composition of claim 1, wherein bedaquiline, or a pharmaceutically acceptable salt thereof is present at from about 73 wt % to about 95 wt % of the total weight of the dry powder composition.

5. The dry powder composition of claim 1, wherein bedaquiline, or a pharmaceutically acceptable salt thereof is present at from about 77 wt % to about 93 wt % of the total weight of the dry powder composition.

6. The dry powder composition of claim 1, wherein bedaquiline, or a pharmaceutically acceptable salt thereof is present at from about 80 wt % to about 90 wt % of the total weight of the dry powder composition.

7. The dry powder composition of claim 1, wherein bedaquiline, or a pharmaceutically acceptable salt thereof is present at from about 85 wt % to about 89 wt % of the total weight of the dry powder composition.

8. The dry powder composition of claim 1, wherein bedaquiline, or a pharmaceutically acceptable salt thereof is present at from about 75 wt % to about 85 wt % of the total weight of the dry powder composition.

9. The dry powder composition of claim 1, wherein bedaquiline, or a pharmaceutically acceptable salt thereof is present at from about 78 wt % to about 82 wt % of the total weight of the dry powder composition.

10. The dry powder composition of claim 1, wherein bedaquiline, or a pharmaceutically acceptable salt thereof is present at from about 79 wt % to about 81 wt % of the total weight of the dry powder composition.

11. The dry powder composition of claim 1, wherein bedaquiline, or a pharmaceutically acceptable salt thereof is present at about 80 wt % of the total weight of the dry powder composition.

12. The dry powder composition of claim 1, wherein bedaquiline, or a pharmaceutically acceptable salt thereof is present at from about 85 wt % to about 95 wt % of the total weight of the dry powder composition.

13. The dry powder composition of claim 1, wherein bedaquiline, or a pharmaceutically acceptable salt thereof is present at from about 88 wt % to about 92 wt % of the total weight of the dry powder composition.

14. The dry powder composition of claim 1, wherein bedaquiline, or a pharmaceutically acceptable salt thereof is present at from about 89 wt % to about 91 wt % of the total weight of the dry powder composition.

15. The dry powder composition of claim 1, wherein bedaquiline, or a pharmaceutically acceptable salt thereof is present at about 90 wt % of the total weight of the dry powder composition.

16. The dry powder composition of any one of claims 1-15, wherein (a) is a pharmaceutically acceptable salt of bedaquiline.

17. The dry powder composition of any one of claims 1-15, wherein (a) is bedaquiline and a pharmaceutically acceptable salt thereof.

18. The dry powder composition of any one of claims 1-15, wherein (a) is bedaquiline.

19. The dry powder composition of any one of claims 1-17, wherein the pharmaceutically acceptable salt of bedaquiline is a pharmaceutically acceptable acid addition salt of bedaquiline.

20. The dry powder composition of any one of claims 1-17 and 19, wherein the pharmaceutically acceptable salt of bedaquiline comprises a fumarate salt of bedaquiline.

21. The dry powder composition of any one of claims 1-17 and 19, wherein the pharmaceutically acceptable salt of bedaquiline is a fumarate salt of bedaquiline.

22. The dry powder composition of claim 20 or 21, wherein the fumarate salt of bedaquiline is (αS, βR)-6-bromo-α-[2-(dimethylamino)ethyl]-2-methoxy-α-1-naphthalenyl-β-phenyl-3-quinolineethanol (2E)-2-butenedioate (1:1) represented by the following formula:

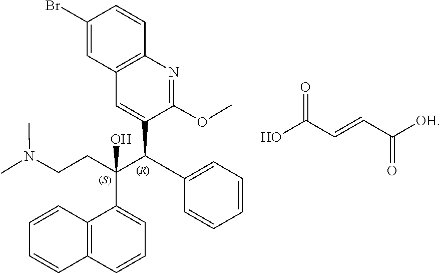

23. The dry powder composition of any one of claims 1-22, wherein the dry powder composition is in the form of an aerosol comprising particles with a mass median aerodynamic diameter (MMAD) of from about 1 μm to about 3 μm, as measured by Next Generation Impactor (NGI).

24. The dry powder composition of any one of claims 1-22, wherein the dry powder composition is in the form of an aerosol comprising particles with an MMAD of from about 1 μm to about 2 μm, as measured by NGI.

25. The dry powder composition of any one of claims 1-22, wherein the dry powder composition is in the form of an aerosol comprising particles with an MMAD of from about 1.5 μm to about 2 μm, as measured by NGI.

26. The dry powder composition of any one of claims 1-22, wherein the dry powder composition is in the form of an aerosol comprising particles with an MMAD of from about 1.5 μm to about 1.8 μm, as measured by NGI.

27. The dry powder composition of any one of claims 1-22, wherein the dry powder composition is in the form of an aerosol comprising particles with an MMAD of about 1.6 μm, as measured by NGI.

28. The dry powder composition of any one of claims 1-27, wherein the dry powder composition is in the form of an aerosol comprising particles with a fine particle fraction of from about 50% to about 75%, as measured by NGI.

29. The dry powder composition of any one of claims 1-27, wherein the dry powder composition is in the form of an aerosol comprising particles with a fine particle fraction of from about 60% to about 75%, as measured by NGI.

30. The dry powder composition of any one of claims 1-27, wherein the dry powder composition is in the form of an aerosol comprising particles with a fine particle fraction of from about 65% to about 75%, as measured by NGI.

31. The dry powder composition of any one of claims 1-27, wherein the dry powder composition is in the form of an aerosol comprising particles with a fine particle fraction of from about 68% to about 72%, as measured by NGI.

32. The dry powder composition of any one of claims 1-27, wherein the dry powder composition is in the form of an aerosol comprising particles with a fine particle fraction of about 71%, as measured by NGI.

33. A method for treating a mycobacterial lung disease in a patient in need of treatment, comprising administering an effective amount of the dry powder composition of any one of claims 1-32 to the lungs of the patient by inhalation via a dry powder inhaler.

34. The method of claim 33, wherein the mycobacterial lung disease is caused by Mycobacterium tuberculosis, Mycobacterium bovis, or a combination thereof.

35. The method of claim 33, wherein the mycobacterial lung disease is caused by Mycobacterium tuberculosis.

36. The method of claim 33, wherein the mycobacterial lung disease is a nontuberculous mycobacterial (NTM) lung disease.

37. The method of claim 36, wherein the NTM lung disease is caused by Mycobacterium abscessus, Mycobacterium asiaticum, Mycobacterium avium, Mycobacterium avium complex (MAC) (Mycobacterium avium and Mycobacterium intracellulare), Mycobacterium avium subsp. hominissuis (MAH), Mycobacterium bolletii, Mycobacterium celatum, Mycobacterium chelonae, Mycobacterium conspicuum, Mycobacterium fortuitum, Mycobacterium fortuitum complex (Mycobacterium fortuitum and Mycobacterium chelonae), Mycobacterium genavense, Mycobacterium gordonae, Mycobacterium haemophilus, Mycobacterium immunogenum, Mycobacterium kansasii, Mycobacterium lentiflavum, Mycobacterium malmoense, Mycobacterium marinum, Mycobacterium mucogenicum, Mycobacterium nonchromogenicum, Mycobacterium peregrinum, Mycobacterium scrofulaceum, Mycobacterium shimoidei, Mycobacterium simiae, Mycobacterium smegmatis, Mycobacterium szulgai, Mycobacterium terrae, Mycobacterium terrae complex, Mycobacterium triplex, Mycobacterium ulcerans, Mycobacterium xenopi, or a combination thereof.

38. The method of claim 36, wherein the NTM lung disease is caused by Mycobacterium abscessus.

39. The method of claim 36, wherein the NTM lung disease is caused by Mycobacterium avium.

40. The method of claim 36, wherein the NTM lung disease is caused by Mycobacterium avium subsp. Hominissuis.

41. The method of claim 36, wherein the NTM lung disease is caused by Mycobacterium avium complex (Mycobacterium avium and Mycobacterium intracellulare).

42. A method for treating lung cancer in a patient in need of treatment, comprising administering an effective amount of the dry powder composition of any one of claims 1-32 to the lungs of the patient by inhalation via a dry powder inhaler.

43. The method of claim 42, wherein the lung cancer is non-small cell lung cancer.

44. The method of claim 42, wherein the lung cancer is small cell lung cancer.

45. The method of claim 42, wherein the lung cancer comprises a lung carcinoid tumor, an adenoid cystic carcinoma, a lymphoma, a sarcoma, a hamartoma, or a combination thereof.

46. A method for treating a metastatic cancer to the lung in a patient in need of treatment, comprising administering an effective amount of the dry powder composition of any one of claims 1-32 to the lungs of the patient by inhalation via a dry powder inhaler.

47. The method of claim 46, wherein the metastatic cancer to the lung comprises metastasis of bladder cancer to the lung.

48. The method of claim 46, wherein the metastatic cancer to the lung comprises metastasis of bone cancer to the lung.

49. The method of claim 46, wherein the metastatic cancer to the lung comprises metastasis of breast cancer to the lung.

50. The method of claim 46, wherein the metastatic cancer to the lung comprises metastasis of colorectal cancer to the lung.

51. The method of claim 46, wherein the metastatic cancer to the lung comprises metastasis of hematopoietic cancer to the lung.

52. The method of claim 46, wherein the metastatic cancer to the lung comprises metastasis of kidney cancer to the lung.

53. The method of claim 46, wherein the metastatic cancer to the lung comprises metastasis of leukemia to the lung.

54. The method of claim 46, wherein the metastatic cancer to the lung comprises metastasis of liver cancer to the lung.

55. The method of claim 46, wherein the metastatic cancer to the lung comprises metastasis of ovarian cancer to the lung.

56. The method of claim 46, wherein the metastatic cancer to the lung comprises metastasis of pancreatic cancer to the lung.

57. The method of claim 46, wherein the metastatic cancer to the lung comprises metastasis of prostate cancer to the lung.

58. The method of claim 46, wherein the metastatic cancer to the lung comprises metastasis of skin cancer to the lung.

59. The method of claim 58, wherein the metastatic cancer to the lung comprises metastasis of melanoma to the lung.

60. The method of claim 46, wherein the metastatic cancer to the lung comprises metastasis of stomach cancer to the lung.

61. The method of claim 46, wherein the metastatic cancer to the lung comprises metastasis of thyroid cancer to the lung.

62. The method of any one of claims 33-61, wherein the administering is conducted in a once-a-day dosing.

63. The method of any one of claims 33-61, wherein the administering is conducted in a twice-a-day dosing.

64. The method of any one of claims 33-61, wherein the administering is conducted in a three-times-a-day dosing.

65. The method of any one of claims 33-64, wherein the administering comprises aerosolizing the dry powder composition and administering an aerosolized dry powder composition to the lungs of the patient by inhalation via the dry powder inhaler.

66. The method of claim 65, wherein the aerosolized dry powder composition comprises particles with an MMAD of from about 1 μm to about 3 μm, as measured by NGI.

67. The method of claim 65, wherein the aerosolized dry powder composition comprises particles with an MMAD of from about 1 μm to about 2 μm, as measured by NGI.

68. The method of claim 65, wherein the aerosolized dry powder composition comprises particles with an MMAD of from about 1.5 μm to about 2 μm, as measured by NGI.

69. The method of claim 65, wherein the aerosolized dry powder composition comprises particles with an MMAD of from about 1.5 μm to about 1.8 μm, as measured by NGI.

70. The method of claim 65, wherein the aerosolized dry powder composition comprises particles with an MMAD of about 1.6 μm, as measured by NGI.

71. The method of any one of claims 65-70, wherein the aerosolized dry powder composition comprises particles with a fine particle fraction of from about 50% to about 75%, as measured by NGI.

72. The method of any one of claims 65-70, wherein the aerosolized dry powder composition comprises particles with a fine particle fraction of from about 60% to about 75%, as measured by NGI.

73. The method of any one of claims 65-70, wherein the aerosolized dry powder composition comprises particles with a fine particle fraction of from about 65% to about 75%, as measured by NGI.

74. The method of any one of claims 65-70, wherein the aerosolized dry powder composition comprises particles with a fine particle fraction of from about 68% to about 72%, as measured by NGI.

75. The method of any one of claims 65-70, wherein the aerosolized dry powder composition comprises particles with a fine particle fraction of about 71%, as measured by NGI.
